(19) United States
(12) Patent Application Publication
(10) Pub. No.: US 2025/0235619 A1
(43) Pub. Date: Jul. 24, 2025

(54) AUTOMATIC INSERTER FOR HANDHELD PEN INJECTION SYSTEM

(71) Applicant: BIOCORP PRODUCTION S.A.S., Issoire (FR)

(72) Inventors: Alain Marcoz, Montmorin (FR); Majid Hihoud, Villette d'Anthon (FR)

(73) Assignee: BIOCORP PRODUCTION S.A.S., Issoire (FR)

(21) Appl. No.: 18/848,688

(22) PCT Filed: Mar. 22, 2022
(86) PCT No.: PCT/IB2022/000141

§ 371 Date: Sep. 19, 2024

(51) Int. Cl. A61M 5/20; A61M 5/31; A61M 5/32; A61M 5/34

(52) U.S. Cl. CPC A61M 5/2033; A61M 5/3137; A61M 5/3202; A61M 5/348; A61M 2005/202

(57) ABSTRACT

An automatic inserter for a pre-filled injection syringe system comprising an elongated body having a longitudinal bore and central longitudinal axis, and configured to receive a pre-filled injection syringe within the bore introduced via a proximal extremity of the body, the inserter further comprising a needle cap holder connected to the distal extremity of the inserter body via an articulation. The cap holder is configured to be rotated about the articulation from a first, closed position, in which it receives the needle cap prior to an injection, to a second, open position in preparation for an injection step, in which the needle cap is removed from the pre-filled injection syringe and held by the cap holder, the latter being connected to the inserter body via the articulation.

### CROSS-REFERENCE TO RELATED APPLICATIONS

This application is a national stage application under 35 U.S.C. § 371 of PCT application No. PCT/IB/2022/000141 filed Mar. 22, 2022 and entitled AUTOMATIC INSERTER FOR HANDHELD PEN INJECTION SYSTEM.

### BACKGROUND

The present invention relates generally to accessories for pre-filled injection syringe systems, and in particular to automatic inserters for pre-filled syringe systems or devices.

Pre-filled injection syringe systems, or also abbreviated to PFS systems or devices, are well known per se and are used to administer a variety of different therapeutic or pharmaceutical products or formulations, often prepared as solutions of varying viscosity. PFS systems commonly comprise a main syringe body forming a chamber in which to contain the pharmaceutical product to be administered, and an internally movable plunger head, located within the syringe body chamber, and which is usually controlled by a plunger shaft connected to the plunger head, and extending out and beyond a proximal, open end of the syringe body. The distal end of the syringe body usually comprises a shoulder and neck portion of narrower diameter than the main syringe body, and a hypodermic needle can be mounted on the neck, or a cannula inserted into the proximal opening of the syringe body, to enable transfer of pharmaceutical product from the chamber of the syringe body into the patient or the subject receiving an injection. It will be understood from what precedes that “proximal” refers to a point directed towards the body or hand of the user that is operating the PFS, and “distal” refers to a point that is directed away from the body of the user of the PFS. Where the user of the PFS and the recipient of the injection are the same, i.e. in the case of self-injection, “proximal” refers to a point in the direction of the hand operating the PFS to effect injection, and “distal” refers to a point directed towards the site of an injection. The syringe body may optionally, and usefully, comprise finger grip means or a prehensile surface such as a flange, located at a proximal end of the syringe body, to enable a user of the PFS to grip the syringe body with two fingers of one hand and push the plunger shaft with the thumb, in a proximal direction and thereby move the plunger head in a proximal direction to cause the product contained in the main syringe body chamber to be ejected through the needle or cannula. The distally located needle or cannula is often coiffed with a removable needle guard or cover, that is typically removed by hand before injection to expose the needle and permit introduction of the needle into an injection recipient's body, whether that part of the body is skin, muscle or some other body part. At this point, and at any time for as long as the needle or cannula is exposed, there is a known risk of accidental exposure to the needle or cannula tip, with corresponding risk of injury, or potentially worse, maladministration of the product contained within the syringe body chamber.

PFS systems of the type described above are used by both medically trained, and non-medically trained users. Whilst medically trained users are accustomed to manipulating such devices after suitable training, individual non-medically trained users, such as the patients themselves, still sometimes struggle to use these PFS devices correctly, and/or appropriately. Manufacturers of such PFS systems have been at pains to attempt to make such systems as easy and foolproof to use as possible and to minimize the risk of accidental injury through the use of such devices. Usually, this has been achieved by hiding to a greater or lesser extent access to the PFS, and devising ways of reducing actual physical contact with the PFS.

Notwithstanding the above developments, there remains, for a number of users at least, difficulties in using such PFS systems correctly, even when they have been hidden by an accessory. For example, some users that need to administer drugs provided in such PFS systems also present with general neural and/or muscular coordination difficulties, rendering the manipulation of the PFS systems and associated accessories incomplete, inexact or imprecise. Other users have a fear of either seeing, handling or otherwise manipulating needles, including inserting the needle into the body, and are therefore faced with a significant psychological challenge when attempting to use the most commonly available commercialized PFS systems, despite the relative improvements in user friendliness. Such challenges can impact a patient's well-being and more importantly, observance of the treatment regime involving such pen injection systems that the user-patient of the device is supposed to be following.

As a result, there have been a few attempts to overcome the above difficulties by providing an automatic, or semi-automatic needle inserter, also known as auto-inserter devices. The aim of such auto-inserter devices, which can be considered both a separate device and an accessory to the PFS in their own right, is to facilitate presentation of the needle of the PFS, at the correct angle for penetration into the body at the site of the injection, and to do so by allowing the user to locate the PFS within the auto-inserter, arm the auto-inserter so that the PFS is ready for injection, and then permit release of the armed injection pen system in a manner which relieves the user from being confronted directly with the sight of having to prick themselves with the needle of the PFS.

For example, published U.S. Pat. Nos. 5,980,491A and 6,537,252 B1, both relate to an automatic insertion device for a pen-shaped injection syringe, the device comprising a tubular housing in which an injection pen is mounted in a tubular pen holder, the pen holder being axially movable along a longitudinal axis of the pen and automatic needle insertion device, in a proximal direction, i.e. towards the hands and/or body of a user, to cock a spring which is thereafter released to drive the pen holder with the pen a set distance in a distal direction. The injection pen is connected distally to a drug containing cartridge, and a needle mounted to the distal end of the drug containing cartridge. This system is specific to the products manufactured and commercialized by applicant of these patents.

Additionally, published French patent application FR3079422A1, relates to another automatic inserter device, configured to receive an injection pen system, such as an insulin injection pen, the auto-inserter device comprising a body with a holder for an injection system, the body being configured to move, via activation of a command member, from an armed position to an unarmed position, thereby enabling axial displacement of the holder. The automatic inserter body comprises a screw-threaded tightening ring system that enables insertion of the injection pen system into the automatic inserter body. The screw-threaded tightening ring system comprises an engagement surface having a proximal frustoconical inner surface which engages progressively via screw threaded action of the tightening ring against a correspondingly shaped frustoconical outer surface of a hollow deformable part, which in turn compresses a plastic ring, thereby reducing the diameter of the opening from a first wider diameter position, in which free axial movement of the pen injection system is permitted, to a second narrower diameter position, in which causes the plastic ring is pressed against, and holds, the body of the pen injection system.

As used herein, the terms “PFS system or device” and “pre-filled syringe system or device” are used interchangeably to designate a generally handheld pre-filled syringe as described generally above, such PFS systems being readily well known per se and commercially available for use in the treatment of many various medical indications. These systems are also often generally designed for self-injection of a drug by the user in need of treatment for the given medical indication. This is for example the case with insulin, supplied in various forms for use in the treatment of diabetes. Similarly configured PFS systems are also available, or have been used, for the treatment of other physiological and/or pathological conditions, using a variety of injectable formulations containing active ingredients such as, for example, adrenaline, epinephrine, methotrexate, recombinant monoclonal antibodies, human growth hormone, hyaluronic acid, and the like.

### SUMMARY

Accordingly, one aspect of the present invention is to provide an automatic inserter for a PFS system which is adapted to receive, and function with the variously shaped and dimensioned pre-filled syringe systems currently in use.

Another aspect of the present invention is to provide an automatic inserter for a PFS system that is easier, safer and more secure to use and handle than the known solutions.

Another aspect of the invention is to provide an automatic inserter for a PFS system that minimizes to a significant extent the possibility for a user of the PFS system to incorrectly prepare and effect an injection from a PFS.

Another aspect of the invention is provide an automatic inserter for a PFS system that reduces the chance of the user accidentally stabbing themselves with the needle.

Another aspect of the invention is to provide an automatic inserter for a PFS system that reduces user fear associated with visible needles.

These and other aspects of the invention will become readily apparent from the complete reading of the current specification.

According to any of the above aspects therefore, there is therefore provided an automatic inserter for a pre-filled syringe system, comprising:
an elongated inserter body having a proximal extremity and a distal extremity, and a longitudinal bore extending through the elongate body from the proximal extremity to the distal extremity, the longitudinal bore having a central longitudinal axis, wherein the elongated inserter body is dimensioned and configured to receive a pre-filled injection syringe introduced into said bore via the proximal extremity of the inserter body, and is further configured and dimensioned to prevent the pre-filled injection syringe from exiting the longitudinal bore via the distal extremity;
wherein the automatic inserter further comprises a needle cap holder connected to the distal extremity of the inserter body via a rotatable articulation, and the needle cap holder is configured to be moved via rotation of the articulation about an axis of rotation of the articulation, from a first, closed position, in which the needle cap holder receives a needle cap removably mounted onto an injection needle of the pre-filled injection syringe when the pre-filled injection syringe is received in the inserter body prior to injection, to a second, open position in preparation for an injection step, in which the needle cap holder holds the needle cap at a location removed from the pre-filled injection syringe, whilst still being connected to the inserter holder body through the rotatable articulation.

As indicated above, the elongated inserter body is dimensioned and configured to receive a pre-filled syringe system introduced into the bore of the inserter body via the proximal extremity thereof. Such a configuration presupposes an opening of the bore at the proximal extremity of the elongated inserter body that is sufficiently dimensioned to allow insertion of at least a distal extremity of the pre-filled syringe into said opening and into the longitudinal bore. Whilst the general overall shape of the elongated inserter body is cylindrical, it is also possible to provide suitably dimensioned, shaped and configured widenings and/or narrowings of the bore along the length of the inserter body.

Furthermore, the elongated inserter body is suitably configured and dimensioned to prevent the pre-filled syringe from exiting the longitudinal bore via the distal extremity of the inserter body. This can be achieved in a variety of ways, for example, by providing one or more abutting shoulders which project inwardly from an inner surface of the elongated inserter body, or other forms of suitable abutment to prevent over insertion of the pre-filled syringe along the axial length of the inserter body or its internally located components situated within the bore. A particularly advantageous solution in this regard is provided in more detail in the present specification.

Additionally, the automatic inserter further comprises a needle cap holder which is connected to the distal extremity of the inserter body via a rotatable articulation. The rotatable articulation can be a simple hinge for example, or an axle or pin and hole mechanism, similar to those commonly found on watch straps, for example. The needle cap holder is movable from a normally closed position, for example, when the inserter is not in use or during introduction of a pre-filled syringe into the inserter body, to an open position in which the needle cap holder no longer closes a distal end opening of the inserter body. The cap holder is moved by rotating the articulation about an axis of rotation. When in the closed position, the needle cap holder receives and retains the needle cap that is generally attached to the distal end of the needle mounted on, or the cannula inserted into, the pre-filled syringe. Such needle caps are commonly found in use with pre-filled syringes to protect the user against accidental stabbing by the needle. As the needle cap holder retains the needle cap in the closed position, when the cap holder is moved to the open position, it temporarily removes the needle cap to a location distant from, or removed from the pre-filled injection syringe, whilst nonetheless remaining connected to the inserter holder body through the rotatable articulation. This is in contrast to specific needle cap removers that are known and which although are configured to receive and remove the needle cap prior to injection, the needle cap remover is generally separated from the pre-filled syringe system, with the disadvantage that the needle cap can be lost, mislaid, or when needed again, and/or incorrectly replaced leading to potential injury from the exposed needle after injection.

According to another aspect, the rotatable articulation has an axis of rotation which lies orthogonal to a central longitudinal axis of the inserter body. In such a configuration, the needle cap holder can be rotated away from, and back towards, the inserter body holding the pre-filled syringe, without interfering in the operation of preparing and effecting the injection.

Accordingly the articulated needle cap holder is also configured to be moved, after an injection has completed, from the second position back to the first position, via rotation of the cap holder about the axis of rotation of the articulation, thereby repositioning the needle cap back onto the injection needle of the pre-filled injection syringe.

According to one aspect, the articulated needle cap holder comprises an elastically deformable friction grip surface configured to engage with, and grip, an outer surface of the needle cap. For example, and advantageously, the friction grip surface comprises an elastomer. The elastomer will be readily chosen from a known list of available elastomers known to provide sufficient friction grip and engaging contact with an outer surface of the needle cap. The grip surface can be provided via a generally V-shaped or open cone shaped set of projections extending from a surface of the needle cap holder that faces inwardly towards the distal opening of the bore of the inserter body. Furthermore, the needle cap holder can also advantageously have a body comprised of the same elastomer, which body will form a generally disc-shaped or circular obscurator to cover the inserter body bore opening located at the distal end of the inserter body. The V-shaped or inverted cone-shaped set of projections are configured to envelope and frictionally engage with the outer surface of the needle cap, which is generally also cone shaped, whereby the nose of the cone of the needle cap will be received and enveloped by the correspondingly cone or V-shaped projections of the needle cap holder.

According to another aspect, the inserter body comprises at least one inner projecting member located within the bore of the inserter body. The inner projecting member can suitably be provided at a distal location within the bore of the inserter body, and can, for example, project into the bore from a side wall of the bore, for example, substantially orthogonally to a longitudinal axis of the inserter body. The inner projecting member is dimensioned and configured to provide a further engagement surface with which the needle cap holder can engage, to ensure that the cap is retained against the bore opening, for example, when the cap holder is in the first, closed position.

According yet another aspect therefore, the articulated needle cap holder comprises, in the first, closed position, an elastically deformable friction grip member configured to elastically deform around, and releasably engage with, the inner projecting member located within the bore of the inserter body. It should be understood that in the second position, the friction grip member of the needle cap holder is free from any engaging contact with the inner projecting member. Such an inner projecting member is therefore also correspondingly free to be moved, if required and/or necessary, or remain in its original position. As will be described hereafter, advantageously, the inner projecting member can be appropriately attached to, or extend from, a movable member located within the bore of the inserter body. In such a configuration, the inner projecting member would thus only be available for the friction grip member of the needle cap holder in certain specific positions of the movable member having such an inner projecting member provided thereon.

According to still another aspect, the elastically deformable friction grip member is formed as an extension of a body of the articulated needle cap holder, and the extension projects into the bore of the inserter body, when the needle cap holder is in the first, closed position.

As mentioned above, the articulated needle cap holder, in the first position, completely covers the distal opening of the inserter body bore, and correspondingly, when the articulated needle cap holder is in the second position, the distal opening of the inserter body bore is left unobstructed by the cap holder and completely open for the injection operations to be prepared and carried out.

According to another aspect, there is provided an automatic inserter for a pre-filled injection syringe system comprising:
an elongated inserter body having a proximal extremity and a distal extremity, and a longitudinal bore extending through the elongate body from the proximal extremity to the distal extremity, the longitudinal bore having a central longitudinal axis, wherein the elongated inserter body is dimensioned and configured to receive a pre-filled injection syringe introduced into said bore via the proximal extremity of the inserter body, and is further configured and dimensioned to prevent the pre-filled injection syringe from exiting the longitudinal bore via the distal extremity;
wherein the inserter body comprises a slidable syringe body carriage assembly located within the bore which is configured to receive and at least partially surround the syringe body,
   wherein the slidable carriage assembly comprises a translation system configured to translate the slidable carriage assembly and syringe body received therein, along a central longitudinal axis of the inserter body bore, from at least a first axial position to at least a second axial position which is axially distant from said first position.

According to this aspect, the inserter body is provided with a slidable syringe body carriage assembly. The slidable carriage assembly is designed and configured to at least partially surround the syringe body, but also be adapted to move the syringe body within and along the longitudinal axis of the bore of the inserter body, so that the syringe body of the pre-filled syringe can be easily positioned at a number of key positions in line with the desired and intended functioning of the automatic inserter. Accordingly, the slidable carriage assembly comprises a translation system configured to translate the slidable carriage assembly and syringe body received therein, along a central longitudinal axis of the inserter body bore, from at least a first axial position to at least a second axial position which is axially distant from said first position. For example, there would be at least two main positions into which it would be desirable to be able to move the slidable carriage assembly holding the syringe body of the pre-filled syringe. A first such position would be an armed position, for example, in which the pre-filled syringe would be placed in a position within the bore of the inserter body enabling it to be accelerated via an appropriately organized translation mechanism such as a spring biased detent, for example. A second such position would be an injection-ready position, for example, to which the pre-filled syringe would have been moved in preparation for the injection step properly speaking, i.e. the step at which the plunger of the pre-filled syringe is operated to expel product from the chamber and into whichever part of the body is the intended target area for administration. Other suitable or advantageous positions for the slidable carriage assembly are also possible and will be discussed in further detail herein.

According then to another aspect, the slidable syringe body carriage assembly comprises a first axially movable cylinder located co-axially within the inserter bore, and a second axially movable cylinder located co axially within the inserter bore, and preferably co axially within the bore of the first movable cylinder. The first and second axially movable cylinders are thus capable of, and are configured to, be moved axially along the central longitudinal axis of the inserter body, one within another, in this case preferably the second cylinder within the first cylinder. Additionally, both first and second cylinders are configured to be movable within the bore of the inserter body.

In accordance with yet another aspect, he first axially movable cylinder and the second axially movable cylinder are configured to be moved axially within the inserter body bore independently one from the other. According to another aspect and as mentioned above, the first axially movable cylinder and the second axially movable cylinder are configured to be moved axially within the inserter body bore together. The reasons for this desired freedom of axial movement of first and second movable cylinders will become apparent from a further reading of the present specification.

According to a further aspect, and as mentioned as being desirable and advantageous above, the at least first axial position is an armed position in which the pre-filled syringe is moved in a proximal direction to arm the pre-filled syringe for a subsequent axial acceleration in a proximal direction prior to an injection step. The armed position is the position in which the automatic inserter pre-positions the pre-filled syringe and accompanying mounted needle thereon, to be moved quickly, or accelerated, from the armed position generally speaking to an axially distant position closer to the intended target or site of administration of the injection. The inserter is called an automatic inserter because this step is equivalent to pulling the trigger on a firearm, for example, and usually involves some kind of stored up energy or detent, for example using biasing elements such as coiled springs. When the stored energy, for example, the springs, are released, usually by activating a trigger mechanism, the released energy is directed in the direction in which the pre-filled syringe and needle are supposed to be accelerated in order to enable the needle to penetrate the administration site, whether that be skin, muscle or some other target. Naturally, the aim of the automatic inserter is to provide only enough directed energy to provide such depth of penetration without pushing the needle in too deep, which would potentially cause pain, and make the user likely to refrain from re-using the device for subsequent injections. The skilled person is capable of providing and designing such energy storage and direction mechanisms, and these are well known per se in the art, for example, in injection pen systems that are commercially available for the treatment of diabetes and other illnesses. Such mechanisms could well be of use in the present case for the automatic inserter.

According then to another object, and as alluded to above, the at least second axial position is an injection-ready position in which at least a distal part of the pre-filled syringe extends beyond a distal end of the inserter body. The length to which the distal part of the pre-filled syringe extends, and in particular to which the needle extends, beyond the end of the inserter body, is designed in such a way as to avoid excessive trauma to the user, and to minimize any associated pain due to the depth of penetration of the needle at the administration site. It should be noted here that the term “injection-ready” only signifies in the current context that the pre-filled syringe is in a suitable position in which to effect the injection of product contained within the syringe chamber, not that an injection has actually been carried out, or activated. The syringe plunger must still be depressed by the user to expel the product from the chamber into the site of administration. This is carried out as a subsequent operation to moving the slidable carriage assembly and pre-filled syringe inserted therein into the injection-ready position.

According to yet another aspect, the inserter body comprises a syringe plunger locking plate. The locking plate serves several purposes, one of which is to lock the plunger against any axial movement with respect to the locking plate, but not prevent axial movement of the locking plate and plunger together in an axial direction.

Thus, another aspect is that the syringe plunger locking plate is configured and adapted to be moved from an unlocked position to a locked position, wherein in the unlocked position, the locking plate is disengaged from the syringe plunger, and in the locked position, the locking plate engages the syringe plunger and prevents axial movement of the syringe plunger relative to the locking plate.

According therefore to one aspect, the locking plate is configured to engage with a proximal end of the syringe body. The locking plate can be advantageously shaped to espouse, envelope, and engage in abutting contact with the proximal end of the syringe body, for example, the finger flange.

According to another aspect, the locking plate can also, for example, and advantageously, be configured to be moved orthogonally to the syringe plunger and the proximal end of the syringe body when moving from the first, unlocked position, to the second, locked position. In such a configuration, the locking plate is disengaged from the syringe plunger and proximal end of the syringe body, when in the first position. It can be moved, from the first, unlocked position, for example, by sliding the plate orthogonally to the central longitudinal axis, along a set of parallel rails or grooves provided transversely to the length of the bore, on an inward facing wall of the inserter body, and towards the proximal end of the syringe body. The locking plate can be advantageously provided, for example, with a flanged groove extending in a distal direction, said flanged groove moving around and espousing and engaging with the finger flange, for example, at the proximal end of the syringe body as it reaches the second, locking position. In the unlocked first position, the locking plate can advantageously present a first end which extends outside the inserter body, whereas in the locked position, the first end is contained completely within the bore of the inserter body. This has the advantage of clearly indicating to the user whether or not the locking plate is engage in the first or second positions, and furthermore, makes it easy to move the locking plate, simply by pushing, or respectively, pulling on the first end of the locking plate to either move it into, or out of, the locked position. Furthermore, only in the locked position is the plate free to be moved within the bore of the inserter body.

In accordance with one aspect therefore, the slidable syringe body carriage assembly comprising the first axially movable cylinder and the second axially movable cylinder are moved together in a proximal direction into the first, armed position, simultaneously with the locking plate. Moving the carriage assembly to the armed position therefore also simultaneously moves the locking plate.

According to another aspect, the moving of the slidable carriage assembly into the first, armed position, causes the needle cap located at the distal extremity of the pre-filled syringe to be retained in the needle cap holder. As has been mentioned above, the needle cap is retained by frictional engagement by the needle cap holder with the needle cap. This frictional engagement is configured to provide greater resistance to separation from the needle cap holder than the corresponding separation resistance between the needle cap and the syringe body shoulder, such that when the syringe body is moved with the slidable carriage into the armed position, i.e. in a proximal direction away from the needle cap holder and the distal end of the inserter body, the needle cap is held by the cap holder and separates from the syringe body shoulder.

Accordingly, the needle cap holder can then be moved from the first, closed position, to the second, open position, when the first axially movable cylinder, second axially movable cylinder, and locking plate are in the first, armed position, as the now exposed needle has been moved in a proximal direction away from any potential contact with the user's hands, thereby avoiding accidental needle-stick injuries.

According to yet another aspect, the slidable syringe body carriage assembly comprising the first axially movable cylinder and the second axially movable cylinder are configured to be moved together in a distal direction into the second, injection-ready position simultaneously with the locking plate. This occurs when the trigger for the energy store is activated. The trigger and energy store form part of the translation mechanism for the automatic inserter, and uses solutions that are known generally to the skilled person, such as biasing springs and the like, to bias the movable cylinders and locking plate into the injection-ready position.

According to another aspect, the second axially movable cylinder of the slidable syringe body carriage assembly is configured to be moved together with the locking plate in a proximal direction to a third, syringe-retracted position. This third, syringe-retracted position only preferably involves the second axially movable cylinder and the locking plate, and corresponds to an axial position along the longitudinal axis of the inserter bore in which the needle no longer extends beyond the distal open end of the inserter bore. In this configuration, were the syringe to be thrown away with the inserter body, then the third position could be a final position into which the syringe would be moved, but as the aim is for the automatic inserter to be re-used, the third position is only an intermediate position, the aim of which is to safeguard the inserter and the user against accidents from a potentially exposed needle after injection has been completed. In this position, the needle of the syringe is entirely contained with the bore of the inserter.

In accordance with another aspect therefore, in the syringe-retracted position, it is possible to move the needle cap holder from the second, open position, to the first, closed position. Doing so will correspondingly move the needle cap back into position for it to be replaced, or remounted, on the distal end of the needle.

According to yet another aspect, the second axially movable cylinder of the slidable syringe body carriage assembly is configured to be moved together with the locking plate in a distal direction to a fourth, syringe-release position. The syringe release position moves the movable cylinders back into a position allowing the now empty or expended syringe to be removed from the inserter bore. Additionally, the locking plate can be slid back to the first, unlocked position, in which the first end of the plate extends beyond the inserter body wall, and furthermore prevent any proximal movement of the cylinders.

As has been mentioned above, with regard to the locking plate, and in accordance with another aspect, the locking plate comprises locking means configured to releasably engage with a proximal end of the second axially movable cylinder of the slidable carriage assembly. Such locking means can for example, be a grooved flange provided on the locking plate, which is shaped to receive, espouse, and engage with, at least a part of the finger flange of the syringe body at the proximal end thereof.

Additionally, and according to yet another aspect, the locking plate comprises a plunger shaft support collar extending in a proximal direction from the locking plate to engage in sliding contact with at least a portion of the plunger shaft, the support collar being configured to move from a collapsed state to a deployed state or from a deployed state to a collapsed state. The support collar serves several purposes, and for example, provides a contact surface for a proximal end of the plunger when injection is being effected. When the proximal end of the plunger comes into contact with the support collar proximally facing contact surface, it prevents excessive force being applied to the movable cylinders.

These and other objects of the invention will become apparent and described in more detail in the following description relating to the figures and an example monitoring module.

### BRIEF DESCRIPTION OF THE FIGURES

The invention will now be described in more detail with regard to the accompanying figures, provided for the purpose of illustration and exemplification, in which:

FIG. 1 is a schematic side representation of an automatic inserter device according the present invention with a pre-filled syringe having been introduced into the inserter;

FIG. 2 is a schematic cross sectional representation of the inserter of FIG. 1;

FIG. 3 is a schematic alternative cross sectional representation of the inserter of FIGS. 1 and 2, rotated about 90°;

FIG. 4 is a schematic top side representation looking along a central longitudinal axis of the inserter of FIG. 1;

FIG. 5 is a schematic perspective representation of the inserter device of FIG. 1;

FIGS. 6A and 6B are schematic cross sectional representations of the configuration of a first relative position of some of the components of the inserter device;

FIGS. 7A and 7B are schematic magnified partial representations of some detail of the components of the inserter device;

FIG. 8 is an exploded perspective representation of the inserter device of FIG. 1, minus any introduced pre-filled syringe;

FIG. 9 is a magnified partial representation of another detail of the inserter device;

FIGS. 10A and 10B are schematic perspective representations of the inserter of FIG. 1, illustrating relative positions of the pre-filled syringe and other components of the inserter;

FIGS. 11A and 11B are schematic cross sectional representations of the inserter as depicted in FIGS. 10A and 10B;

FIG. 12 is a magnified partial representation of another detail of the inserter device;

FIG. 13 is a magnified partial representation of a different detail of the inserter device;

FIGS. 14A and 14B are schematic cross sectional representations of some of the relative positions of the components of the inserter device;

FIG. 15 is a schematic cross sectional representation of the inserter device in an injection-ready position;

FIG. 16 is a schematic cross sectional representation of the inserter device depicted in FIG. 15, in an injection-ready position and rotated about 90°;

FIG. 17 is a magnified partial representation of a detail of the inserter device;

FIG. 18 is a schematic cross-sectional representation of the relative position of the syringe within the inserter in a retracted position;

FIG. 19 is a further schematic cross-sectional representation of the relative position of the syringe within the inserter in a retracted position showing additional detail;

FIG. 20 is a schematic cross-sectional representation of the relative position of the syringe within the inserter in another position;

FIG. 21 is a schematic cross-sectional representation of the relative position of the syringe after withdrawal from the inserter after use.

### DETAILED DESCRIPTION OF ILLUSTRATIVE EMBODIMENTS

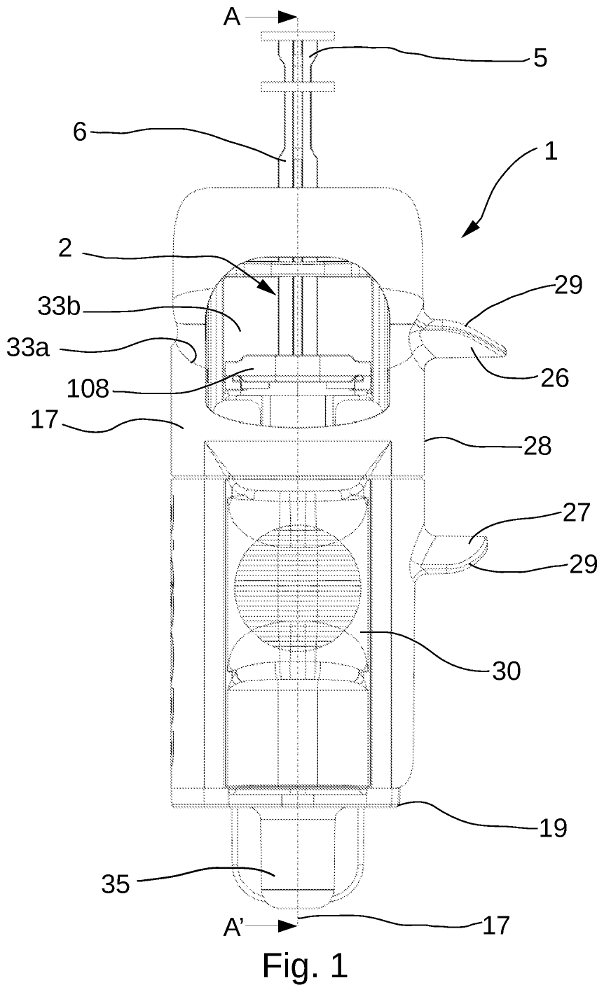

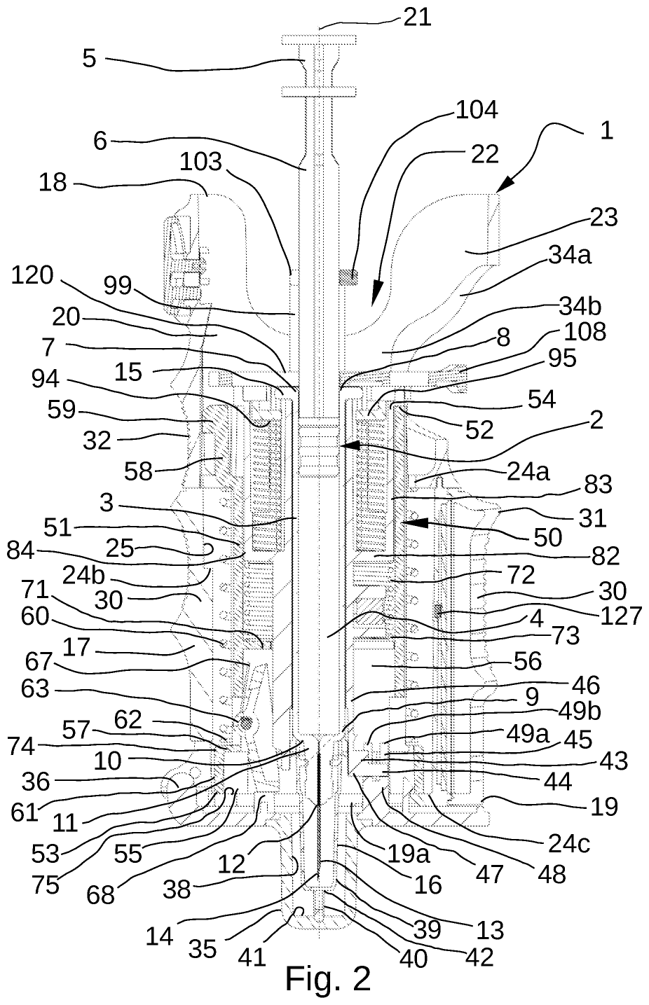

Turning now to the figures, FIGS. 1 and 2 schematically illustrate a side view (FIG. 1) and a cross-sectional representation (FIG. 2) along the axis A-A′, i.e. from the left hand side of FIG. 1, of an automatic inserter device (1) according the present invention with a pre-filled syringe (2) having been introduced into the inserter. The pre-filled syringe (2), or “PFS system or device”, or “PFS” for short, as used in the present specification designates a generally handheld pre-filled syringe, such PFS systems being readily well known per se and commercially available for use in the treatment of many various medical indications. PFS systems commonly comprise a main syringe body (3) forming a chamber (4), cf. FIG. 2, in which to contain the pharmaceutical product to be administered, and an internally movable plunger head (5), located within the syringe body chamber (4), and which is usually controlled by a plunger shaft (6) connected to the plunger head (5), and extending out and beyond a proximal end (7) of the syringe body (3) having an opening (8). The distal end (9) of the syringe body (3) usually comprises a shoulder (10) and neck (11) portion of narrower diameter than the main syringe body (3), and a distal opening (12), wherein a hypodermic needle (13) having an open distal end (14) can be mounted on the neck (11), for example via a needle mount connector which engages with the neck (11) and shoulder (10) in a known manner, e.g. via a screw threading, or a cannula inserted into the distal opening (12), to enable transfer of pharmaceutical product from the chamber (4) of the syringe body (3) into the patient or the subject receiving an injection. It will be understood from what precedes that “proximal” refers to a point generally directed towards the body or hand of the user that is operating the PFS, and “distal” refers to a point that is directed away from the body of the user of the PFS. Where the user of the PFS and the recipient of the injection are the same, i.e. in the case of self-injection, “proximal” refers to a point in the direction of the hand operating the PFS to effect injection, and “distal” refers to a point directed towards the site of an injection. The syringe body (3) may optionally, and usefully, comprise finger grip means or a prehensile surface such as a flange (15), located at the proximal end (7) of the syringe body (3), to enable a user of the PFS to grip the syringe body (3) with two fingers of one hand and push the plunger head (5) with the thumb, in a proximal direction, and thereby move the plunger shaft (6) in a distal direction to cause the product contained in the main syringe body chamber (3) to be ejected through the distal opening (14) of the needle (13) or cannula. The distally located needle (13) or cannula is often coiffed with a removable needle guard (16) or cover, that is typically removed by hand before injection to expose the needle (13) and permit introduction of the needle into an injection recipient's body, whether that part of the body is skin, muscle or some other body part. At this point, and at any time for as long as the needle (13) or cannula is exposed, there is a known risk of accidental exposure to the needle (13) or cannula tip, with corresponding risk of injury, or potentially worse, maladministration of the product contained within the syringe body chamber (3).

The automatic inserter device (1) as illustrated in the figures comprises an elongated inserter body (17) having a proximal extremity (18) and a distal extremity (19), and a longitudinal bore (20) extending through the elongate body (17) from the proximal extremity (18) to the distal extremity (19), the longitudinal bore (20) having a central longitudinal axis (21), wherein the elongated inserter body (17) is dimensioned and configured to receive the pre-filled injection syringe (2) introduced into the bore (20) via the proximal extremity (18) of the inserter body (17) along the central longitudinal axis (21). Such a configuration presupposes an opening (22) of the bore (20) at the proximal extremity (18) of the elongated inserter body (17) that is sufficiently dimensioned to allow insertion of at least the distal end (9) of the pre-filled syringe (2) into said opening and into the longitudinal bore (20). Whilst the general overall shape of the elongated inserter body (17) is cylindrical, it can be seen from FIGS. 1 and 2 that the body also comprises widenings (23) and/or narrowings (24) of the bore along the length of the inserter body (17). The narrowings (24) can, for example, be suitably formed as one or more abutting shoulders (24a, 24b, 24c, etc) which project inwardly from an inner surface (25) of the elongated inserter body, or other forms of suitable abutment to prevent over-insertion of the pre-filled syringe along the axial length of the inserter body or other internally located components situated within the bore, as will be described herein.

The automatic inserter device (1) also comprises actuatable positioning means, locking means and stabilizing means to maintain the PFS (2) in a series of predetermined axial and lateral positions which operate according to a sequence, thereby restricting relative axial movement of the PFS within the inserter device (1) and, additionally, preventing it from exiting the longitudinal bore (20) via the distal extremity (19) of the inserter body (17). The details of these actuatable positioning means, locking means and stabilizing means will be provided hereafter, but as illustrated in FIGS. 1 and 2, the PFS (2) is held in a first, locked, axial and lateral position, wherein the lateral position locking means relates to means for preventing the PFS from moving or being moved, laterally away from, and out of alignment with, the central longitudinal axis (21). As shown in FIG. 1, the automatic inserter device elongated body (17) is provided with a first prehensile member (26) and a second prehensile member (27) spaced apart from the first prehensile member (26), along the longitudinal axis of (21) the inserter body (17) and located on, or projecting outwardly from, an outward facing surface (28) of the inserter body (17). The first prehensile member (26) and the second prehensile member (27) are shaped and configured respectively to facilitate grasping of the inserter device (1) by a user. The distance between the first prehensile member (26) and second prehensile member (27) is configured to allow comfortable resting of one or more fingers against an outward facing surface (29) of the first (26) and/or second (27) prehensile member during use of the inserter device (1). As a complement to the first and second prehensile members (26, 27), an area or region (30) of increased grip is located on the outward facing surface (28) of the inserter body (17), for example located at 90° or 180°, about the longitudinal axis (21), in relation to the prehensile members (26, 27). The area of increased grip (30) can suitably be provided by a series of appropriately shaped ridges (31) and troughs (32) comprised of a soft molded elastane material, such materials being known per se. As can also be seen from FIG. 1, the elongated inserter body (17) has at least one, or more, cut-away (33a, 33b) portions which define openings (34a, 34b) in the elongated inserter body (17), located adjacent, or in proximity, to the proximal end (18) of the elongated inserter body (17). The cut-away portions or openings (33a, 33b, 34a, 34b) allow the user to see, and/or access some of the components of the inserter device and/or the PFS (2) located within the bore (20), and also provide user access, and visibility, to a locking mechanism for locking the PFS (2) into a predefined initial axial position within the longitudinal bore (20) of the inserter body (17) as will be described in more detail hereafter.

The automatic inserter device (1) further comprises a needle cap holder (35) connected to the distal extremity (19) of the inserter body (17) via a rotatable articulation (36). The needle cap holder (35) is configured to be moved via rotation of the articulation (36) about an axis of rotation (37) of the articulation (36), from a first, closed position, in which the needle cap holder (35) receives a needle cap or needle guard (16) removably mounted onto the injection needle (14) of the PFS (2), when the latter is received in the inserter body (17) prior to injection, to a second, open position, in preparation for an injection step, in which the needle cap holder (35) holds the needle cap (16) at a location removed from the PFS (2), whilst still being connected to the inserter holder body (17) through the rotatable articulation (36). The rotatable articulation (36) can be a simple hinge for example, or an axle or pin and hole mechanism, similar to those commonly found on watch straps, for example. The needle cap holder (35) is therefore movable from a normally closed position, for example, when the inserter device (1) is not in use, or during introduction of a PFS (2) into the inserter body (17), to an open position in which the needle cap holder (35) no longer closes the distal end (19) opening (19a) of the inserter body (17). The cap holder (35) is moved by rotating the articulation (36) about the axis of rotation (37) of the articulation (36). When in the closed position, the needle cap holder (35) receives and retains the needle cap (16) that is attached to the distal end (14) of the needle (13) mounted on, or the cannula inserted into, the pre-filled syringe (2). Such needle caps (16) are commonly found in use with pre-filled syringes to protect the user against accidental stabbing by the needle. As the needle cap holder (35) retains the needle cap (16) in the first, closed position, when the cap holder (35) is moved to the second, open position, it temporarily removes the needle cap (16) to a location distant from, or removed from the pre-filled injection syringe (2), whilst nonetheless remaining connected to the inserter holder body (17) through the rotatable articulation (36). This is in contrast to known needle cap removers, which although they are configured to receive and remove a needle cap prior to injection, such known needle cap removers are generally separated from the pre-filled syringe system, with the disadvantage that the needle cap can be lost, mislaid, or when needed again, incorrectly replaced, thereby leading to potential injury from the exposed needle after injection.

As is apparent from the figures, the rotatable articulation (36) has an axis of rotation (37) which lies orthogonal to the central longitudinal axis (21) of the inserter body (17). In such a configuration, the needle cap holder (35) can be rotated away from, and back towards, the inserter body (17) holding the pre-filled syringe (2), without interfering in the operation of preparing and effecting the injection. Accordingly the articulated needle cap holder (35) can be moved, after an injection has completed, from the second, open position back to the first, closed position, via rotation of the cap holder (35) about the axis of rotation (36) of the articulation (37), thereby repositioning the needle cap (16) back onto the distal end opening (14) of the injection needle (13) of the pre-filled injection syringe (2).

The articulated needle cap holder (35) also advantageously comprises an elastically deformable friction grip surface (38) configured to engage with, and grip, an outer surface (39) of the needle cap (16). For example, the friction grip surface (38) comprises an elastomer. The elastomer will be readily chosen from a known list of available elastomers known to provide sufficient friction grip and engaging contact with the outer surface (39) of the needle cap (16). The friction grip surface (38) can be provided via a generally V-shaped or open cone shaped set of projections (40) extending from a surface (41) of the needle cap holder (35) that faces inwardly towards the distal opening (19a) of the bore (20) of the inserter body (17). The needle cap holder (35) advantageously also comprises a body (42) comprised of the same elastomer as the friction grip surface (38), which body (42) will form a generally disc-shaped or circular obscurator to cover the inserter body bore opening (19a) located at the distal end (19) of the inserter body (17). The V-shaped or inverted cone-shaped set of projections (40) are configured to envelope and frictionally engage with the outer surface (39) of the needle cap (16), which is generally also cone shaped, whereby the nose of the cone of the needle cap (16) will be received and enveloped by the correspondingly cone or V-shaped projections (40) of the needle cap holder (35).

The inserter body comprises at least one inner projecting member (43) located within the bore (20) of the inserter body (17). The inner projecting member (43) can suitably be provided at a distal location within the bore (20) of the inserter bold (17), and can, for example, project into the bore (20) from a side wall of the bore (20), substantially orthogonally to a longitudinal axis of the inserter body (17), or from another component located within the bore (20) as will be described herein in more detail. The inner projecting member (43) is dimensioned and configured to provide an engagement surface (44) with which the needle cap holder (35) can engage, to ensure that the cap holder (35) is retained against the bore opening (19a), for example, when the cap holder (35) is in the first, closed position. In the first, closed position of the needle cap holder (35), the projecting member (43), as illustrated in FIG. 2, engages with an elastically deformable friction grip member (45) configured to elastically deform around, and releasably engage with, the inner projecting member (43) located within the bore (20) of the inserter body (17). It should be understood that in the second, open position, the friction grip member (45) of the needle cap holder (35) is free from any engaging contact with the inner projecting member (43). Such an inner projecting member (43) is therefore also correspondingly free to be moved, if required and/or necessary, or remain in its original position. The inner projecting member (43) is advantageously attached to, or extends from, a movable member (46) located co-axially within the bore of the inserter body (17). In such a configuration, the inner projecting member (43) would thus only be available for the friction grip member (45) of the needle cap holder (35) in certain specific positions of the movable member (46) having such an inner projecting member (43) provided thereon. FIG. 2 illustrates what such a projecting member (43) can look like, in which said projecting member (43) is an orthogonal spar, extending outwards from a distal end (47) of an axially movable syringe body (3) holder (46), which syringe body holder (46) is co-axially located inside the bore (20) of the elongated inserter body (17). As can also be seen from FIG. 2, the elastically deformable friction grip member (45) is formed as an extension (48) of the body (42) of the articulated needle cap holder (35), and the friction grip member extension (48) projects in parallel to the central longitudinal axis into the bore (20) of the inserter body (17), when the needle cap holder (35) is in the first, closed position. The extension (48) bears at least one pair of elastically deformable arms (49a, 49b) at a proximal end of the extension (48), which extend further in a proximal direction from the friction grip member extension (48), each pair of arms (49a, 49b) engaging with a respective side surface (44a, 44b) of the orthogonally positioned projecting member (43). As mentioned above, the articulated needle cap holder (35), in the first position, completely covers the distal opening (19a) of the inserter body bore (20), and correspondingly, when the articulated needle cap holder (35) is in the second position, the distal opening (19a) of the inserter body bore is left unobstructed by the cap holder (35) and completely open for the injection operations to be prepared and carried out.

Furthermore, as can be seen from FIG. 2, the automatic inserter device (1) also comprises a slidable syringe body carriage assembly (50) located co-axially around the central longitudinal axis (21) and within the bore (20) of the elongated inserter body (17). The slidable syringe body carriage assembly (50) is configured to receive and at least partially surround the syringe body, via the syringe body holder (46). The slidable carriage assembly (50) comprises a translation system configured to translate the slidable carriage assembly (50) and syringe body (3) received therein, along the central longitudinal axis (21) of the inserter body bore (20), from at least a first axial position to at least a second axial position which is axially distant from said first position.

The slidable carriage assembly (50) is designed and configured to at least partially surround the syringe body (3), but also be adapted to move the syringe body (3) within and along the longitudinal axis (21) of the bore of the inserter body (17), so that the syringe body (3) of the pre-filled syringe (2) can be easily positioned at a number of key positions in line with the desired and intended functioning of the automatic inserter device (1). For example, there are be at least two main positions into which it would be desirable to be able to move the slidable carriage assembly (50) holding the syringe body (3) of the pre-filled syringe. A first such position is an armed position, for example, in which the pre-filled syringe (2) is located in a position within the bore (20) of the inserter body (17) enabling it to be accelerated via an appropriately organized translation mechanism such as a spring-biased detent, for example. A second such position is an injection-ready position, for example, to which the pre-filled syringe (2) would have been moved in preparation for the injection step properly speaking, i.e. the step at which the plunger of the pre-filled syringe is operated to expel product from the chamber (4) and into whichever part of the body is the intended target area for administration. Other suitable or advantageous positions for the slidable carriage assembly are also possible and will be discussed in further detail herein.

The slidable syringe body carriage assembly (50) comprises a first axially movable cylinder (51), illustrated in FIG. 2 as an outer slider body (51) which is located co-axially within the inserter bore (20), and a second axially movable cylinder (46), illustrated in FIG. 2 as an internal slider body (46) or syringe holder body (46), and which is located co-axially within the inserter bore (20), and also co-axially within the bore of the outer slider body (51). Outer and inner slider bodies (51, 46) are movable axially along the central longitudinal axis (21) of the inserter body (17), the latter within the former, either together or independently one from the another, as the need arises, in order to move one or the other of the axially movable cylinders into one or more positions which are relevant to the use of the inserter device (1).

The first axially movable cylinder, or outer slider body (51), extends from a proximal end (52) located in a proximal area of the bore (20) towards a distal end (53) and has a proximal opening (54) and a distal opening (55) forming a longitudinal bore (56). Near the distal end (53), the outer slider body has a first radially outwardly extending shoulder (57). Near the proximal end (52), the outer slider body (51) also has an elastically deformable arm (58), which extends radially outwardly from the cylinder body, and which terminates in a proximal hook (59), the functioning of which will be described further herein. Between the radially outwardly extending shoulder (57) and the elastically deformable arm (58) a biasing spring (60) is located around an outward-facing surface (61) of the outer slider body (51), the biasing spring (60) engaging with the radially outwardly extending shoulder (57) at a distal end (62) of the spring (60). Situated proximally of the radially outwardly extending shoulder (57), the outer slider body (51) is provided with a lever arm safety lock (63), the functioning of which will be described further herein. The lever arm safety lock (63) has a pivoting locking arm (64) which has a proximal arm member (64a) and a distal arm member (64b), the proximal and distal arm members (64a, 64b) extending from a fulcrum pivot point (65) mounted on a hinge bar (66), said hinge bar (66) forming an axis of rotation similar to an elbow about which the arm (64) pivots from a first, unlocked position to a second, locked position, under the impetus of a biasing spring (67), and in reverse, from the second, locked position to the first, unlocked position, under the impetus of a contact spigot (68) extending axially into the bore (56) from the needle cap holder (35). When the needle cap holder is in the first, closed position, and the outer cylinder is in a rest position, the contact spigot (68) comes into engaging contact with a shoulder (69) extending substantially orthogonally with regard to the longitudinal axis (21) from a distal end (70) of the distal arm (64b). The proximally directed force of the spigot (68) against the shoulder (69) is transmitted to the distal arm locking member (64b), causing the arm as a whole (64) to pivot about the fulcrum point (65) and thereby forcing the proximal arm locking member to be biased against the biasing spring (67). This is then the unlocked position of the lever arm safety lock. The biasing strength of the biasing spring (67) is chosen to not be so strong as to overcome the lever force exerted about the fulcrum due to contact between the spigot (68) and shoulder (69). However, once the contact between the spigot (68) and shoulder (69) is removed, for example, when the needle cap holder is removed, the biasing spring (67) no longer encounters any resistance and will tend to bias the proximal arm locking member (64b) towards the center of the bore (56), and towards the first, locked position, as will be described further herein. The outer slider body (51) also has at least one radially inwardly projecting shoulder (71), located proximally of the lever arm safety lock, and against which is seated another biasing spring (72) at a distal end (73) of the spring (72). The inner biasing spring (72) of the outer slider also comes into compression or biasing contact with the inner slider, or syringe holder body (46), detailed further herein. Finally, the outer slider body (51) extends in a distal direction from an outer edge (74) of the radially outwardly extending shoulder (57) to the distal end (53), which distal end (53) is in abutting contact with a distal end surface (75) of the inserter body (17), to prevent the outer cylinder (51) from being moved beyond the distal end (19) of the inserter body (17).

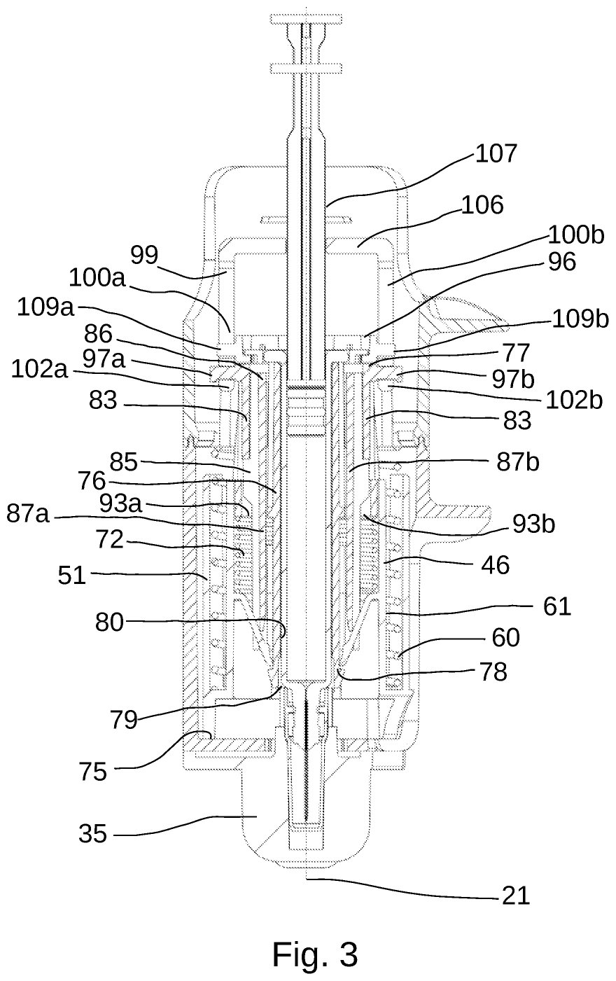

As shown in FIGS. 2 and 3, in which FIG. 3 represents a cross-section view of FIG. 1, i.e. cutting through the face of automatic inserter device of FIG. 1, the inner slider body, or syringe body holder (46) is located co-axially within the outer slider body (51). The inner slider body (46) is a mainly tube-like cylindrical body (76) which extends from a proximal end (77) to a distal end (78) and which defines a central longitudinal bore (79) having an inward facing surface (80). The inward facing surface (80) is shaped and dimensioned to receive, and engage with, a corresponding outward facing surface of the syringe body (3). Movement of the inner slidable body (46) in a distal direction is restricted by the inner biasing spring (72) of the outer slidable body (51), the inner slidable body (46) sitting on the inner biasing spring (72) in the initial rest position, as illustrated in FIG. 2. The inner slidable body (46) is provided with a radially outwardly extending shoulder (82), and a peripheral annular wall (83) extending in a proximal direction from an outside edge (84) of the shoulder (82). The peripheral annular wall (83) and tube-like cylindrical body (76) define an annular space (85) extending in parallel to the longitudinal axis from the proximal end (77) of the inner slider body (46) to the radially outwardly extending shoulder (82). A sliding caliper (86) having a first leg (87a) and a diametrically opposing second leg (87b) is received in the annular space (85), each caliper leg being provided with an outwardly facing, longitudinally oriented, groove (88a, 88b) running along the length of the legs (87a, 87b), each groove receiving a respectively corresponding biasing spring (89a, 89b). The biasing springs (89a, 89b) sit on a proximal-facing surface (90) of the radially outwardly extending shoulder (82). The first caliper leg (87a) and second caliper leg (87b) are connected at respective proximal ends (91a, 91b) of the respective legs (87a, 87b) to a cylindrical caliper body (92), which is dimensioned to fit, and slide along the longitudinal axis (21), within the annular space (85). The legs (87a, 87b) of the caliper (86) extend from the proximal ends (90a, 90b) in a distal direction through a pair of respective openings (93a, 93b) provided in the radially outwardly extending shoulder of the inner slider (46) and into the bore (56) of the outer slider (51), and consequently in parallel to, and outside of, the tube-like body (76) of the inner slider (46). The biasing springs (89a, 89b) therefore sit on the bottom of the annular space (85) in a position which is between a respective opening (93a, 93b) of the radially extending shoulder (82) and the peripheral annular wall (86), and are received in a respective groove (88a, 88b) of the caliper legs (87a, 87b). The biasing springs (88a, 88b) also engage with, and are constrained or compressed by, a distal-facing surface (94) of a proximally located radially-inwardly extending shoulder (95), which extends radially inwards from the cylindrical caliper body (92) into the inserter body bore (20). The cylindrical caliper body (92) is also provided, at a proximal end (96) of the caliper body, with a pair of diametrically opposed outwardly projecting spars (97a, 97b). The spars are shaped and configured to receive, and function as an axis of rotation (98) for, a plunger shaft support collar (99). The support collar (99), at its distal end (100), is mounted rotatingly or pivotally on the caliper body (92) via the spars (97a, 97b) which extend through, and provide a respective axis of rotation (98) for, a respective and corresponding pair of support collar legs (100a, 100b), which have a transverse opening (101a, 101b) at the distal end (102a, 102b) of each of the legs (100a, 100b). Each support collar leg (100a, 100b) extends from the distal ends (102a, 102b) in a proximal direction towards a proximal transverse bar (103) which connects the legs (100a, 100b) together at the proximal end (104) of the support collar (99). The transverse bar (103) is provided, substantially in a center part thereof, with a curved, or arcuate cradle (105) centered around the bore (20), and which is shaped and dimensioned to engage in abutting and supporting, or cradling, contact with the outside surface of the plunger shaft (6) when the PFS (2) is introduced into the automatic inserter device (1) and during operation of the automatic inserter device (1). The arcuate cradle (105) of the support collar (99) is shaped to allow the plunger shaft (6) to slide axially in the cradle (105) in both a distal and a proximal direction, depending on the relative movements imparted by the other movable components of the automatic inserter device (1), as will be described herein in further detail. The support collar (99) is furthermore configured to move from a collapsed state, in which the collar lies in a plane which is orthogonal to the central longitudinal axis (21), and is substantially in a horizontal position, to a deployed state in which the support collar is substantially in a vertical position, in parallel alignment with the central longitudinal axis, and vice-versa, to be able to return to the collapsed state. The support collar (99) serves several purposes, and for example, provides a proximal contact surface (106) for a proximal end (107) of the plunger shaft (6), when injection is being effected. During distal axial movement of the inner and outer sliders (46, 51), for example, during injection, contact between the proximal end (107) of the plunger shaft (6) and the proximally facing contact surface (106) of the support collar (99) prevents excessive force being applied to the inner and outer slidable cylinders (46, 51), and thereby reduces the risk of damage to the overall integrity of the inserter device. As will be understood from the foregoing, the support collar (99) is connected to the caliper (92), which in turn can slide or translate axially within the annular space (85) of the inner slider body (46), the caliper legs (87a, 87b) extending through the openings (93a, 93b) in a distal direction as the caliper is moved in an axial direction.

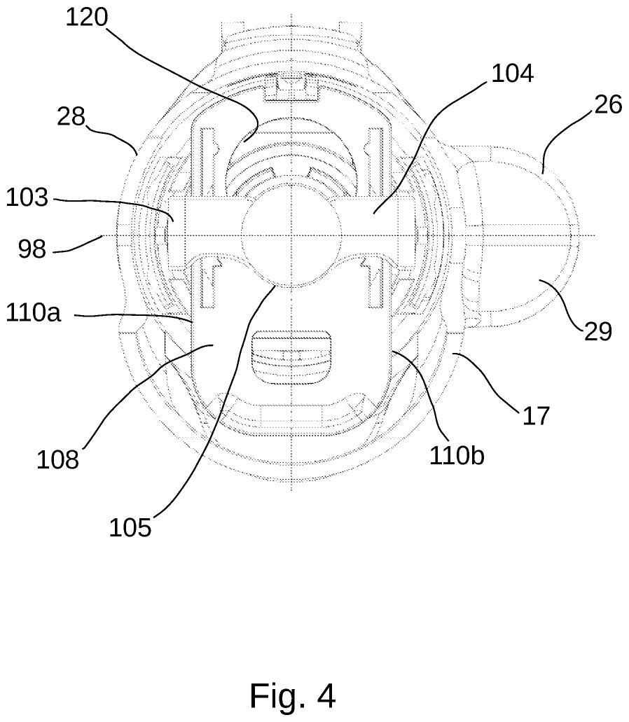

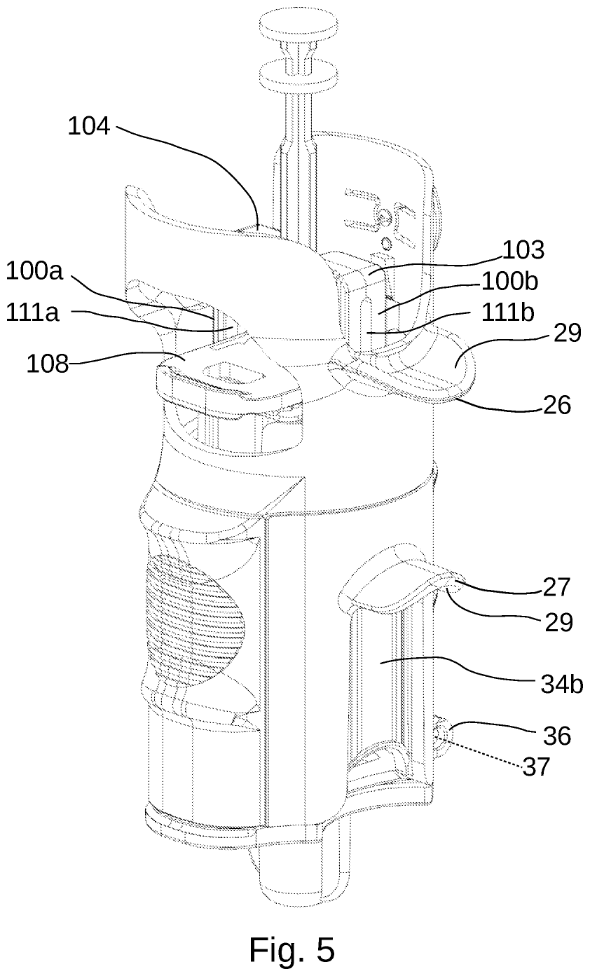

As illustrated in FIGS. 2, 3 and in the top plan view of FIG. 4 a syringe plunger locking plate (108) is connected to the legs (100a, 100b) of the support collar (99) via a pair of diametrically opposed spigots (109a, 109b) extending outwardly from respective opposing lateral sides (110a, 110b) of the plunger locking plate (108). Each spigot (109a, 109b) engages with a respective correspondingly dimensioned longitudinal slot (111a, 111b) provided in the legs (100a, 100b) of the support collar (99). The locking plate spigots (109a, 109b) are dimensioned to be able to slide along, and be guided by, the slots (111a, 111b). The locking plate is movable from a first unlocked position, to a second locked position. In the unlocked position, the locking plate (108) is disengaged from the syringe body (6) and the syringe plunger shaft (6), and in the locked position, the locking plate engages both the syringe body (3) and the syringe plunger shaft (6), thereby prevents axial movement of the syringe body (3) and plunger shaft (6) relative to the locking plate. The locking plate (108) is located orthogonally to the central longitudinal axis (21), and is moved orthogonally, or laterally, with respect to the central longitudinal axis, when the user presses on the plate (108) from an exposed, or open part (34a, 34b) of the inserter body (17), which opening is located at a proximal narrowing (24) of the inserter body (17). The locking plate (108) is configured to engage with the proximal end (77) of the inner slider body (46). To that end, the locking plate (108) is provided, on a distal-facing surface (112) of the locking plate, with a pair of parallel, spaced apart and oppositely facing, L-shaped projections (113a, 113b) which form a pair of opposing grooves (114a, 114b), extending along the distal facing surface (112) along a respective lateral side (110a, 110b) and orthogonally relative to the central longitudinal axis (21). The projections (113a, 113b) forming the locking plate grooves (114a, 114b) are dimensioned to engage with the diameter and dimensions of the proximal end (77) of the inner slider body (46), as the locking plate (108) is moved laterally from the unlocked position to the locked position. The grooves (113a, 113b) formed by the L-shaped projections (113a, 113b) engage with, and surround, a proximal lip, or flange (115), both on a proximal-facing surface (116), and a distal-facing surface (117) of the lip (115), which extends around a periphery of the proximal end (77) of the inner slider (46). The distal-facing surface (112) of the locking plate is further provided with at least one, and preferably two, pairs of abutment stops (118a, 118b, 118c, 118d) which extend from the L-shaped projections. The abutment stops (118a, 118b, 118c, 118d) are configured, positioned and dimensioned to engage with and abut against corresponding counter-stops (119a, 119b, 119c, 119d) provided on the distal-facing surface (117) of the flange (115) of the proximal end (77) of the inner slidable body (46). The stops and counter-stops (prevent the locking plate being moved too far laterally, and also provide audible feedback to the user when the locking plate (108) has been moved into the correct locking position, thereby securing the proximal end (77) of the syringe body (3) within the inner slidable body (46). The locking plate further comprises an essentially circular opening (120) which traverses the thickness of the locking plate, and which is dimensioned, in the unlocked position, to allow introduction of the syringe body (3) and plunger shaft (6) of the PFS (2) into the automatic inserter device bore (20). Adjacent to, and forming a continuous union intersection with, the locking plate opening (120) a subsidiary opening (121) is provided, of dimensions corresponding to those of the diameter of plunger shaft (6). Thus, when the locking plate is moved laterally out of the unlocked position into the locked position, so the opening (120) is moved laterally and the smaller, subsidiary opening (121) is moved into the locked position to engage with the outer diameter of the plunger shaft (6). In this way, the locking plate (108) serves several purposes, one of which is to lock the plunger shaft (6), in the locked position, against any lateral movement away from the central longitudinal axis (21) with respect to the locking plate (108), and also prevent unwanted axial movement of the syringe body relative to the other axially movable components of the inserter device (1). Additionally, as the locking plate is moved from the unlocked position into the locked position, the spigots (109a, 109b) move within the guide slots (111a, 111b) of the support collar legs (100a, 100b). The support collar legs (111a, 111b) are attached via the distal openings (101a, 101b) at their respective distal ends (102a, 102b) to the caliper spars (97) and therefore to the fixed point axis of rotation (98). Thus, as the locking plate is moved laterally from the unlocked position to the locked position, and the spigots of the locking plate slide in, and engage with, the leg slots (111a, 111b), the plunger support collar (99) is rotated about the axis (98) from the collapsed position, in which the collar (99) is substantially horizontal, to the deployed position, in which the collar (99) is substantially vertical, thereby bringing the collar cradle (105) into engaging cradling contact with the plunger shaft (6). An overall representation of this state is given by the perspective view of the automatic inserter device including the locked PFS (2) as illustrated in FIG. 5.

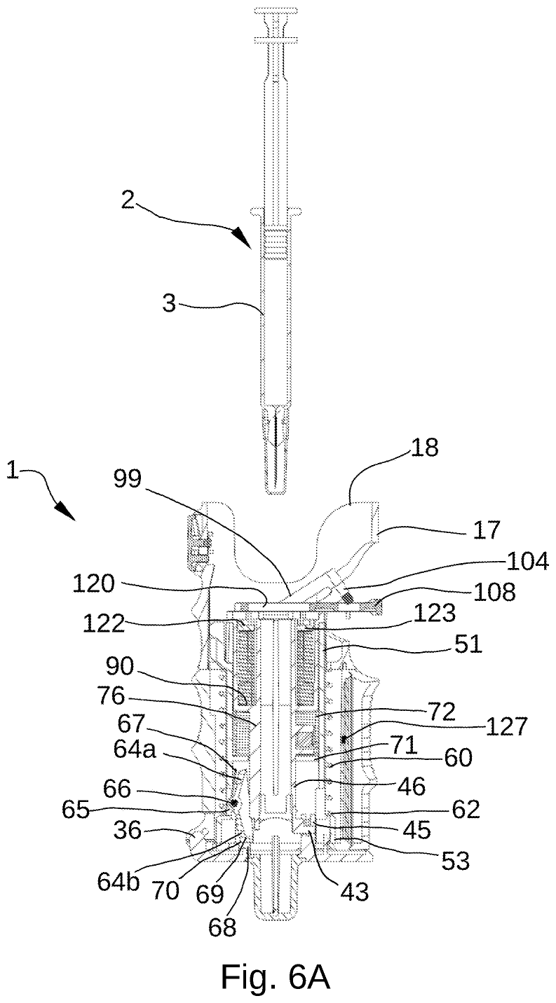

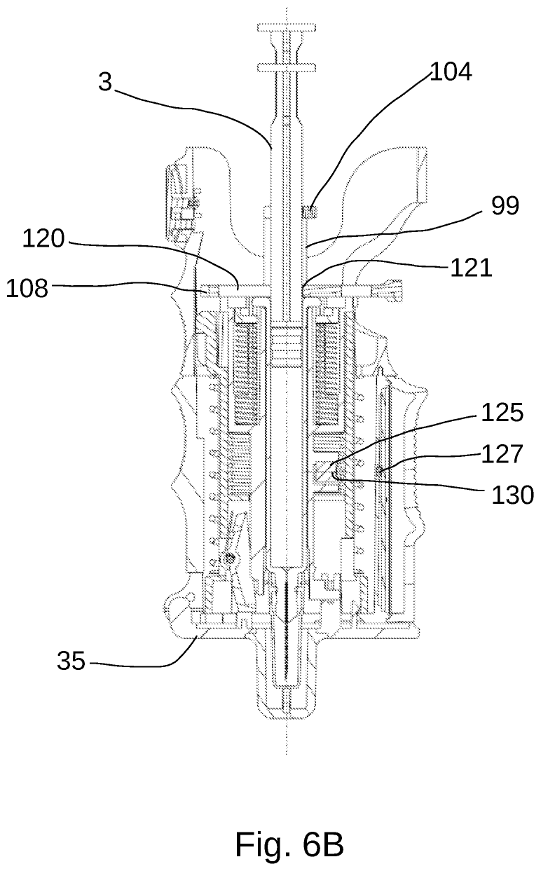

A brief description of the operation of the device is now provided, referring the reader to FIGS. 6A and 6B, with regard to introduction of the PFS (2) into the automatic inserter device (1) and the bringing of the PFS (2) into an initial, or rest position. The PFS (2) is introduced, needle end first, into the inserter device via the open, proximal end (18) of the inserter body (17) and through the opening (120) of the locking plate (108), which is in the unlocked position, into the bore of the tube-like body (76) of the inner slider body (46). The tube-like body (76) receives and engages with the outer surface of the syringe body (3). The syringe body is prevented from being inserted distally too far into the tube-like body (76) of the inner slider body due to the general dimensions of the tube-like body which are designed and configured to mate with the outer diameter of the syringe body (3), but additionally, due to a proximal seat (122) formed by a radially, inwardly extending shoulder (123) provided at the proximal end of the tube-like body (76). The seat (122) also serves to locate and center the syringe body (3) within the bore of the tube-like body of the inner slider body (46). The locking plate (108) is now moved laterally by the user, for example by pressing with the thumb, it is moved from right to left, orthogonally across the longitudinal axis (21) of the inserter device (1). The locking plate (108) movement thereby causes the L-shape projections (113a, 113b) and corresponding locking grooves (114a, 114b) engage with, and surround the proximal-facing (116) and distal-facing (117) surfaces of the proximal flange or lip (115) of the inner slidable body (46). When the locking plate has reached the locking position, an audible signal, such as a click, is produced by the interaction between the abutment stops (118a, 118b, 118c, 118d) of the locking plate (108) and the counter-stops (119a, 119b, 119c, 119d) located on the distal-facing surface of the proximal end of the inner slider body (46). Additionally, as the locking plate is moved laterally from the unlocked to the locked position, so the support collar (99) is moved from a horizontal, collapsed, position to a vertical, deployed, position, in which the support collar (99) cradle engages with the plunger shaft (6). Similarly, the locking plate opening (120) is moved laterally by the lateral movement of the locking plate (108) to cause the subsidiary opening (121) to be brought into central axial alignment and consequently an engaging contact with the plunger shaft (6). In the locked position of the locking plate (108), the syringe body (3) is held between the seat (122) located distally of the syringe finger flange (15), and the distal surface of the locking plate (108) proximally of the syringe finger flange (15), and is therefore incapable of axial movement independently of the other axially movable components.

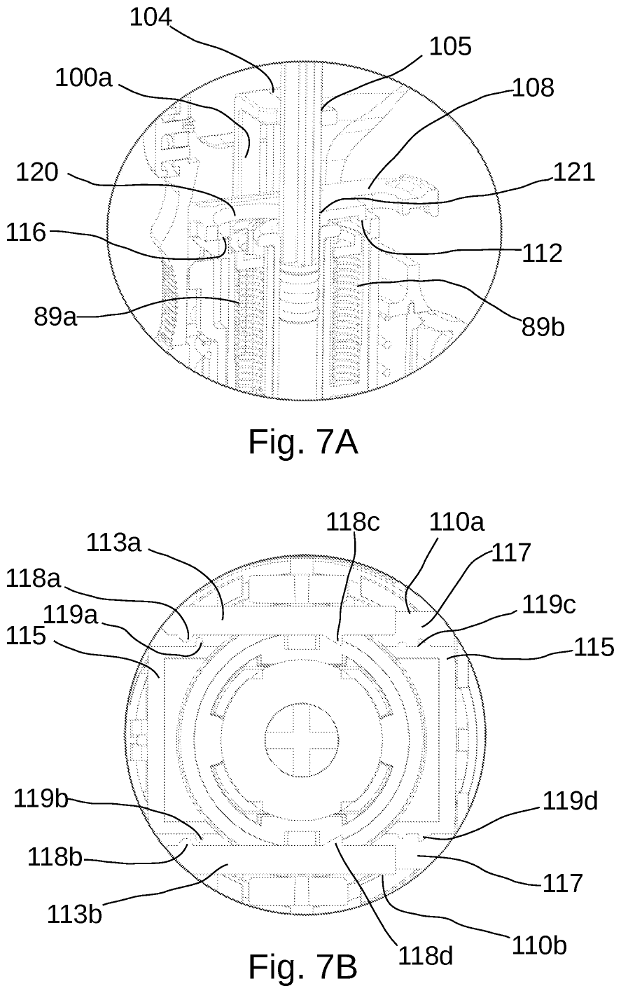

FIG. 7A illustrates a partially cut-away magnified perspective view of the various details of the locking plate (108) in the locked position relative to the support collar (99), the plunger shaft (6) and syringe body (3) of the PFS (2), as they would be located in the initial, rest position. Similarly, FIG. 7B shows a magnified bottom plan view along the central longitudinal axis (21) and illustrating the relative detail of the abutment stops (118a, 118b, 118c, 118d) and counter-stops (119a, 119b, 119c, 119d) as the locking plate (108) is being moved from the unlocked position to the locked position. As can been seen from FIG. 7B, the locking plate abutment stops (118a, 118b, 118c, 118d) extend from the L-shaped projections (113a, 113b). As the locking plate is moved from the unlocked to the locked position, which in FIG. 7B is from left to right, the abutment stops (118a, 118b) move into abutting contact with the counter-stops (119a, 119b). Continued applied lateral pressure to the locking plate by the user causes the abutment stops (118a, 118b) to be forced past the counter-stops (119a, 119b) until abutment stops (118c, 118d) encounter counter-stops (119c, 119d), at which point an audible click sound is generated as the abutment stops (118c, 118d) snap into place between counter-stops (119c, 119d). The locking plate (108) is now considered to be in the locked position.

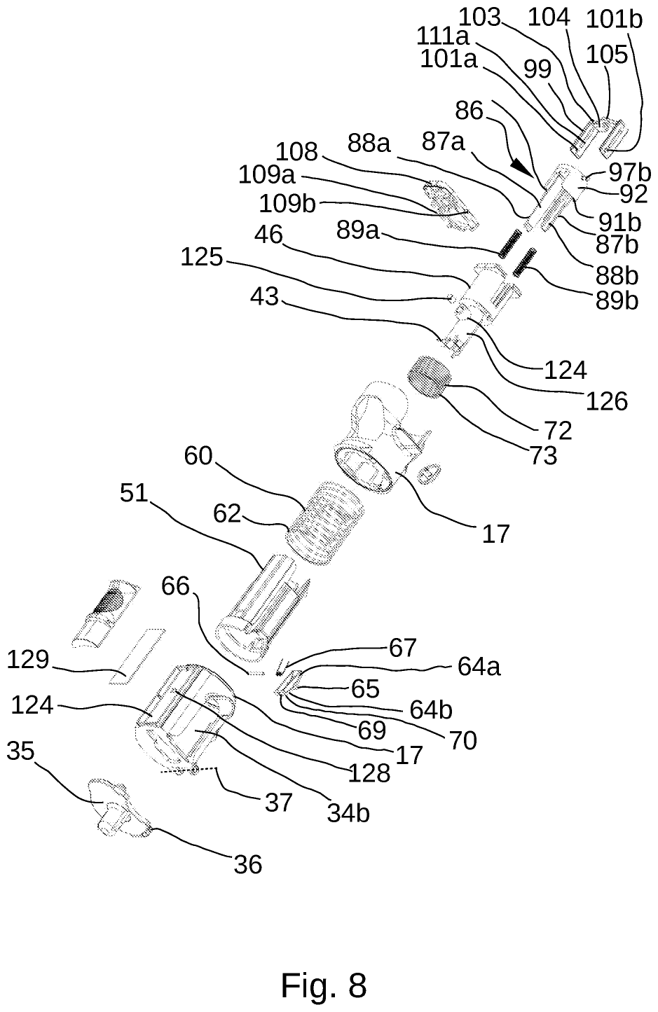

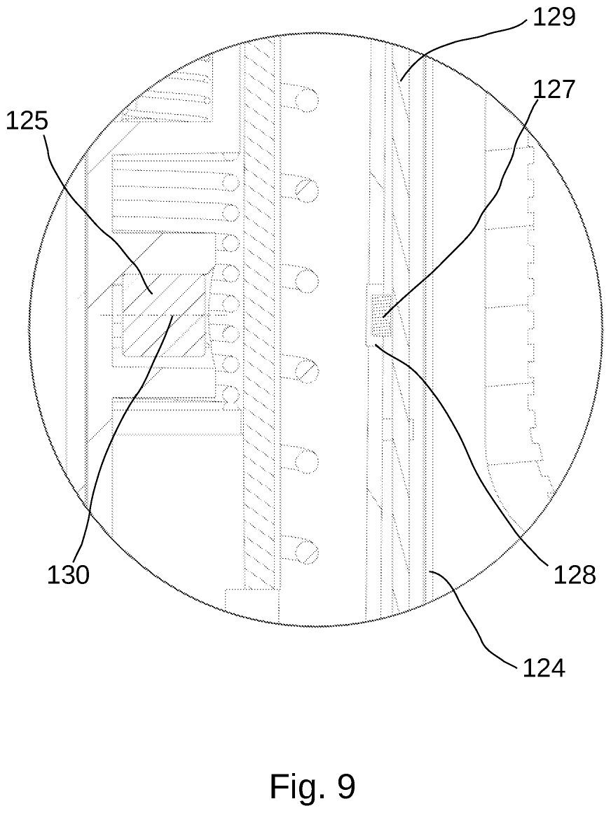

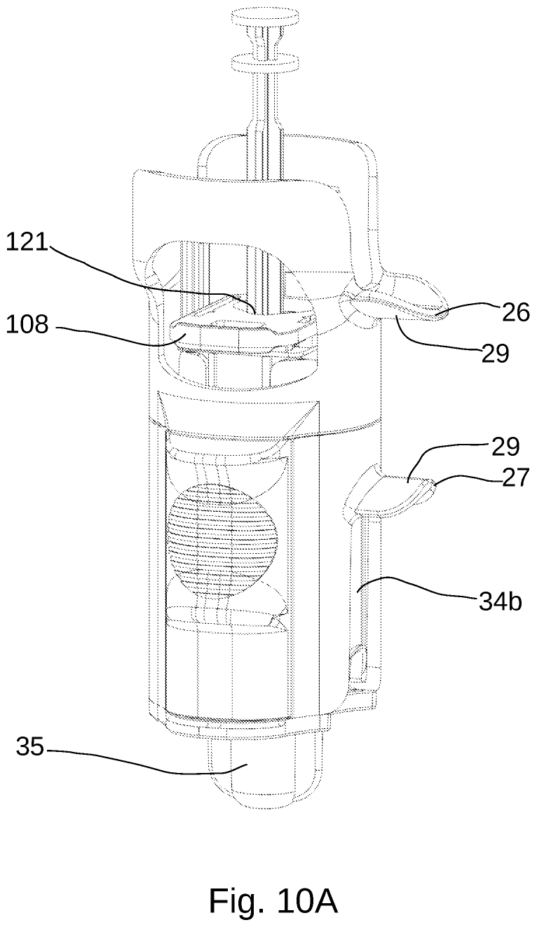

FIG. 8 illustrates an exploded perspective view of the automatic inserter device (1) without any pre-filled syringe, in which the various components of the inserter device are disposed along, or either side of, the central longitudinal axis (21). For the sake of convenience, like elements have been identified with identical numbers to the elements in FIGS. 1 to 4, with the following further components being displayed. From FIG. 6, it can be seen that the inner slider body (46) has a position signaling element housing (124) for receiving a position signaling element (125), which housing projects from, and is located on, an outward-facing surface (126) of the slider body (46), in a position that is both distal to the radially outwardly extending shoulder (82), and at 900 about the central longitudinal axis (21) relative to the openings (93a, 93b) in the radially outwardly extending shoulder (82). The position signaling element (125) located within the position signaling element housing (124) can be any appropriate signaling element, which when used in combination with a position sensor (127), communicates, or allows the determination, or the calculation of, the position of the inner slidable body (46) with respect to the component in which the position sensor (127) is housed. In the present example, the position signaling element (125) is usefully a permanent or inducible N-S dipole magnet, for example a disk or rod magnet having a single N-S dipole. The position sensor (127) is correspondingly, and advantageously, a magnetometer, although, it should be bome in mind that other known pairings of position signaling element and position sensor could be used to similar effect. The position sensor (127) is housed on a micro-electronic circuit board (129), such as a printed circuit including a micro-controller, for example including a built-in processing unit, which is electrically or otherwise functionally connected to the position sensor (127). In FIG. 8, for example, the position sensor (127) is appropriately included in a micro-electronic printed circuit board (129) which is located in a position sensor housing (128) provided in a distal section of the automatic inserter device body (17). For example, FIGS. 2, 6A, 6B, and FIG. 9 in magnified detail, show the relative positions of the position signaling element (125) and the position sensor (127), along with a nominal center line (130) extending orthogonally through the position signaling element (125), which nominal center line is deemed to represent position point zero, i.e. the initial rest position when the PFS (2) has been introduced into the automatic inserter device (1) and locked into the locked position. Any changes in registered magnetic field or magnetic vector values picked up, or determined, by the sensor (127) from the position signaling element (125) will be indicative of axial movement of the inner slider body (46), and outer slider body (51) when or if the latter is locked in axial displacement with the inner slider body (46).

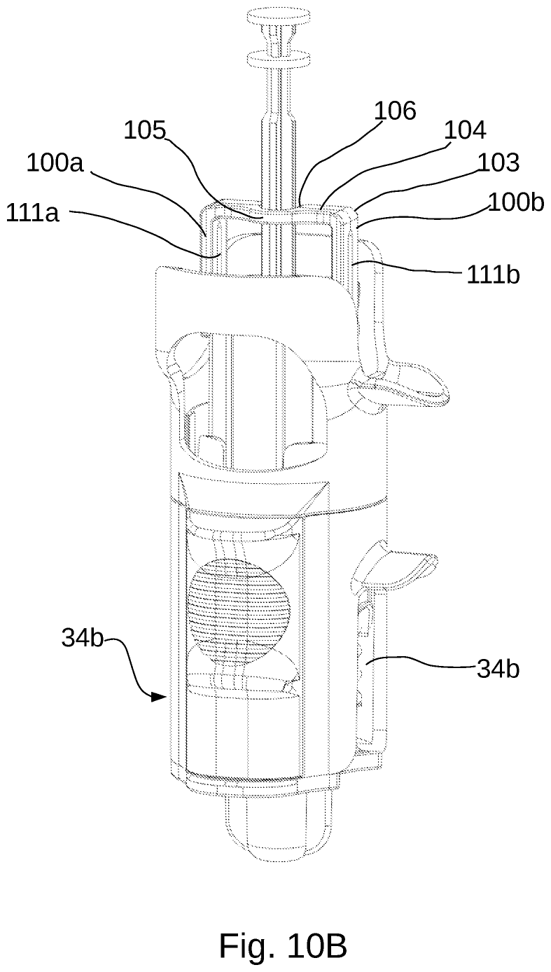

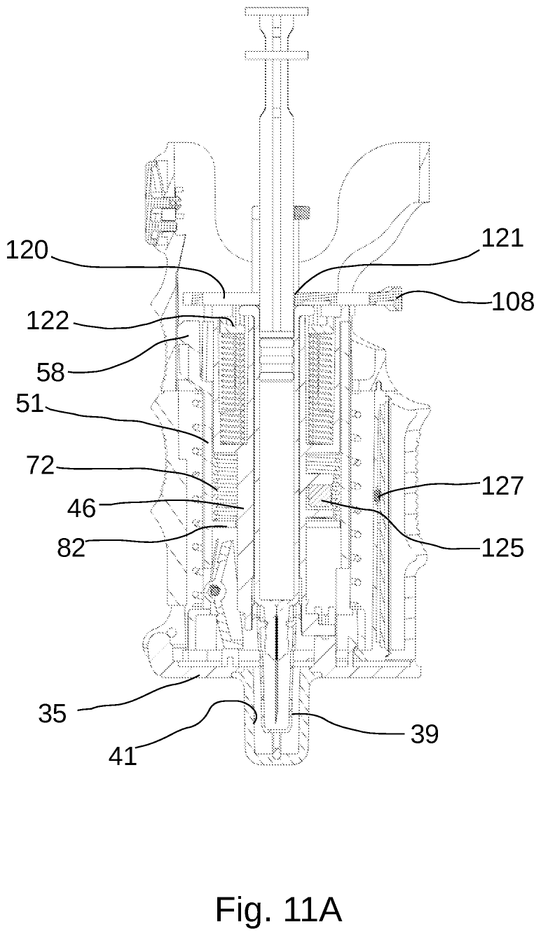

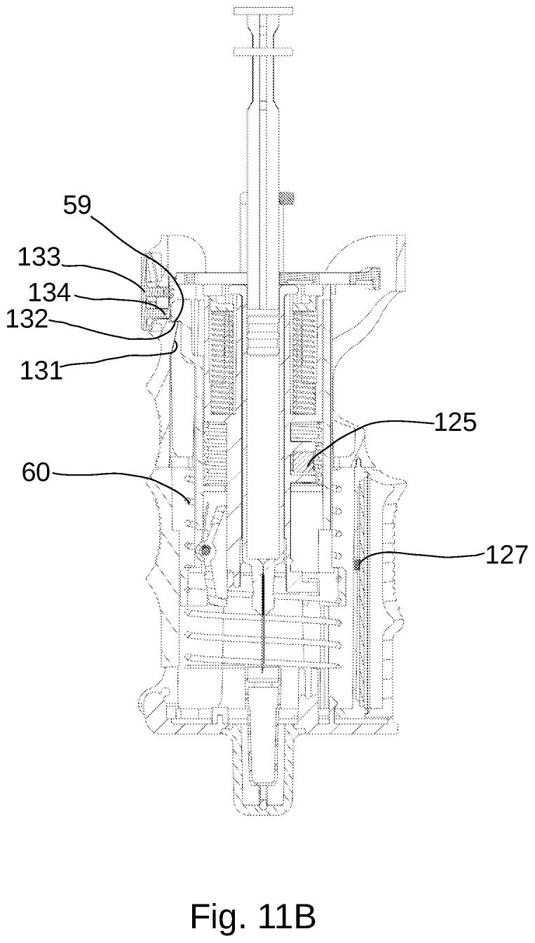

FIGS. 10A, 10B, 11A, and 11B are comparative representations in perspective and cross-section of the automatic inserter device (1) and relative positions of the PFS (2), plunger support collar (99), and plunger shaft (6), when moving from the initial rest position, or position zero, to the first, armed position. For example, it can be seen in FIGS. 10B and 11B that the support collar (99) in the armed position has been moved in a proximal direction well beyond the proximal end (18) of the inserter body (17). The armed position is a position in which the pre-filled syringe (2) is moved in a proximal direction to arm the pre-filled syringe (2) for a subsequent axial acceleration in a distal direction prior to an injection step. The armed position is the position in which the automatic inserter device (1) pre-positions the pre-filled syringe (2) and accompanying mounted needle (13) thereon, to be moved quickly, or accelerated, from a proximally located, armed position to a distally located axial position closer to the intended target or site of administration of the injection. The inserter device (1) is called an automatic inserter because the step of activating the inserter device (1) to move from the armed position to the distal position ready for injection is equivalent to pulling the trigger on a firearm, for example, and usually involves a stored up energy source or detent, for example using biasing elements, and as illustrated in the Figures, such as coiled springs. When the stored energy, i.e. the springs, are released, usually by activating a trigger mechanism, the released energy is directed in the direction in which the pre-filled syringe and needle are supposed to be accelerated in order to enable the needle to penetrate the administration site, whether that be skin, muscle or some other target. Naturally, the aim of the automatic inserter device (1) is to provide only enough directed energy to provide such depth of penetration without pushing the needle in too deep, which would potentially cause pain, and make the user likely to refrain from re-using the device for subsequent injections. The skilled person is capable of providing and designing such energy storage and direction mechanisms, and these are well known per se in the art, for example, in injection pen systems that are commercially available for the treatment of diabetes and other illnesses.

To move the automatic inserter device (1) from the initial rest position, the user moves the distal ends (53) of the outer slidable body (51) in a proximal direction, for example by pulling on them, or pushing them. The distal ends (53) of the outer slidable body (51) are exposed via the distal openings (34b) in the inserter body (17) and are therefore available for manipulation by the user. In FIG. 11A, one can see, for example, that the position signaling element (125) is aligned with the position sensor element (127). At the same time as the user moves the outer slider body (51) in the proximal direction, it also seizes the needle cap holder (35) on the outer needle cap surface (39) by the cap holder friction grip surface (38), thereby forcing the inward facing surface (41) of the needle cap holder (35) to grip the needle cap (16). The needle cap (16) is retained by the needle cap holder (35) as the outer slider body (51) is moved in the proximal direction because the radially inwardly projecting shoulder (71) of the outer slider body (51) causes compression of the inner biasing spring (72) of the outer body slider (51), which in turn biases against the radially outwardly extending shoulder (82) of inner slider body (46), also in the proximal direction. The consequence of this chain biasing is that both the outer slider body (51) and inner slider body (46) are biased in the proximal direction, and move the PFS (2) in the same direction, as the latter is trapped in the locked position by the locking plate (108) and the proximal seat (122) of the tube-like body (76) of the inner slider body (46). The locking plate (108) is therefore also moved in the proximal direction along with the support collar (99) and outer and inner slider bodies (51, 46). The total distance of travel in the proximal direction of the outer body from the initial rest position is about 20 mm. As the outer slider body (51) is moved in the proximal direction, the elastically deformable arm (58) is moved axially to same extent in the proximal direction. When the arm starts to reach the maximum allowed distance of travel of the outer slider body in the proximal direction, the elastically deformable arm (58) is caused to deform elastically inwards into the outer body bore as it is urged against a sloping segment (131, FIG. 11B) with a hooked proximal end (132) of the inserter body (17), which sloping segment projects into the inserter body bore (20). The elastic deformation of the arm (58) is configured to allow the arm (58) to overcome the resistance urged on by the sloping segment and hooked proximal end (132), at which point the elastically deformable arm (58) snaps back outwards towards the inserter body (17) and the proximal terminal hook (59) of the deformable arm engages with the hooked proximal end (132) of the inserter body (17) to prevent the outer slider body (51) from being urged in a distal direction until such time as the inserter device needs to be operated to move the device from the armed position to the injection-ready position. In the armed position, the position signaling element (125) is the maximum distance from the position sensor (127), and therefore the generated signal at its weakest, in the case where the signaling element is a magnet, for example. The microelectronic circuitry is thus capable of identifying when the automatic inserter device (1) has been moved into the armed position.

The release of the automatic inserter device (1) can be achieved via an elastically deformable release button (133), advantageously located on, and housed by, the inserter body (17) near the proximal end (18) of the inserter body. The elastically deformable release button (133) is provided with a projecting biasing spar (134) which extends through the wall of the inserter body and is positioned in alignment with the terminal hook (59) of the elastically deformable arm (58). When release from the armed position to the injection ready position is desired, the user presses on the release button (133), which deforms elastically inwards and causes the projecting spar (134) to move inwards into biasing contact with the terminal hook (59), pushing said hook out (59) and deforming the elastically deformable arm such that the terminal hook (59) loses contact with the hooked proximal end (132) of the sloping segment (131). When this occurs, the pent up energy stored by the biased and compressed outer biasing spring (60) of the outer slider body (51) causes the outer slider body (51), inner slider body (46), locking plate (108), support collar (99) and the PFS (2) to be urged in a distal direction into the injection ready position.

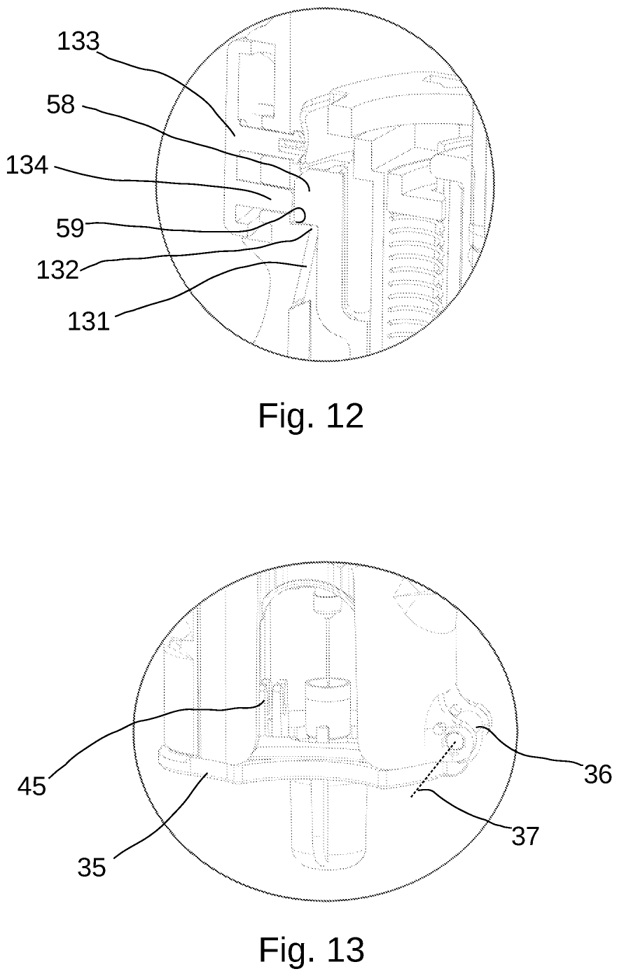

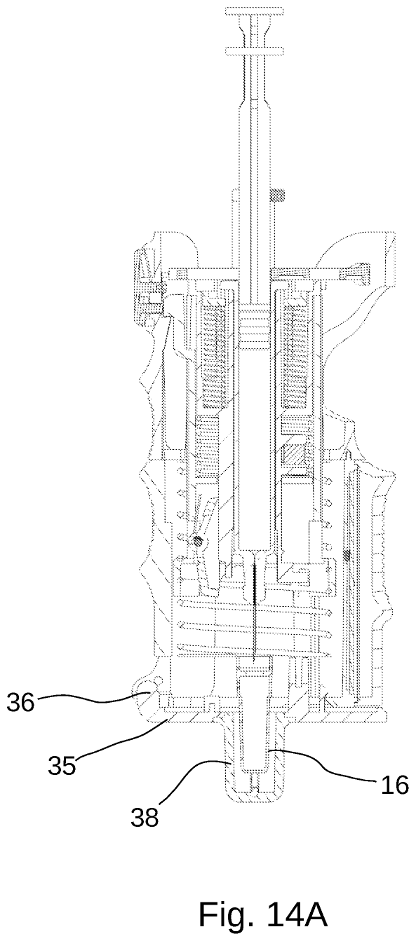

In regard to the above, FIG. 12 shows a magnified, partially cut-away view of the relative positions of the deformable elastic arm (58) and terminal hook (59) with the sloping segment (131) and hooked proximal end (132), the elastically deformable release button (133) and projecting spar (134). FIG. 13 shows a magnified view of the release of the needle cap (16) from the distal needle end (14) as the outer slider body (51) is moved in the proximal direction, and the protection that is still offered against accidental injection due to the presence of the needle cap holder (35) which has retained the needle cap (16).

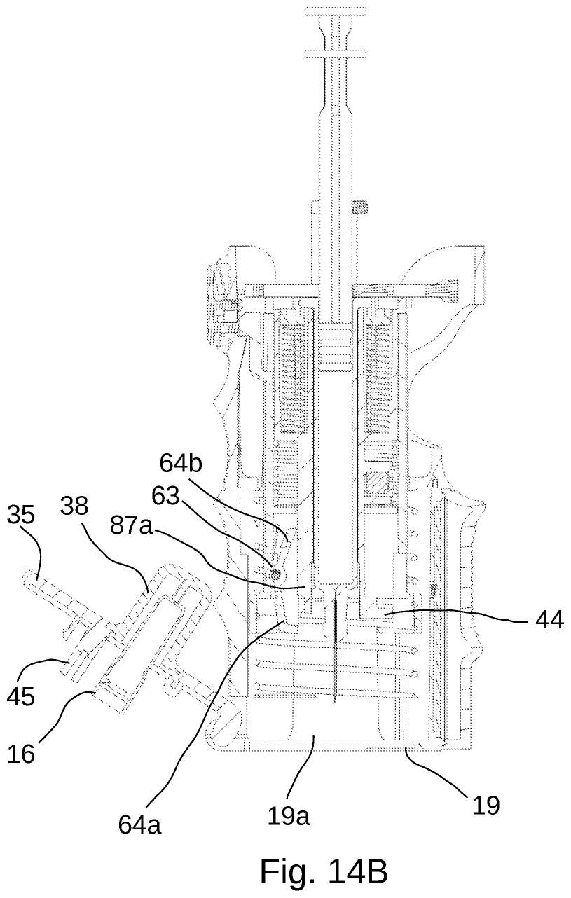

FIGS. 14A and 14B illustrate the relative positions of the components of the inserter device (1) between the release from the armed position and the injection ready position. In order to prepare the inserter device (1) for release from the armed position, the user must first remove the needle cap holder (35) which is holding the needle cap (16). This is achieved by pulling on the needle cap holder friction grip surface (38), which causes the needle cap holder to rotate about rotatable articulation (36) and corresponding axis of rotation (37) to move the needle cap holder away from the distal end (19) of the inserter device body (17), as clearly illustrated in FIG. 14B. It should also be noted that when the outer slider body (51) is moved in the proximal direction into the armed position, the distal locking arm member (64b) is no longer biased by the needle cap holder (45), and as a result, the proximal locking arm member (64a) of the lever arm safety lock (63) is biased by the lever arm biasing spring (67), causing the lever arm to rotate into the bore (56) and the proximal locking arm member (64a) to be urged against one of the caliper legs (87a). Similarly, the proximal movement of the inner slider body during arming of the inserter device (1), causes the inner projecting member engagement surface (44a, 44b) to move away from engaging contact with the elastically deformable friction grip member (45).

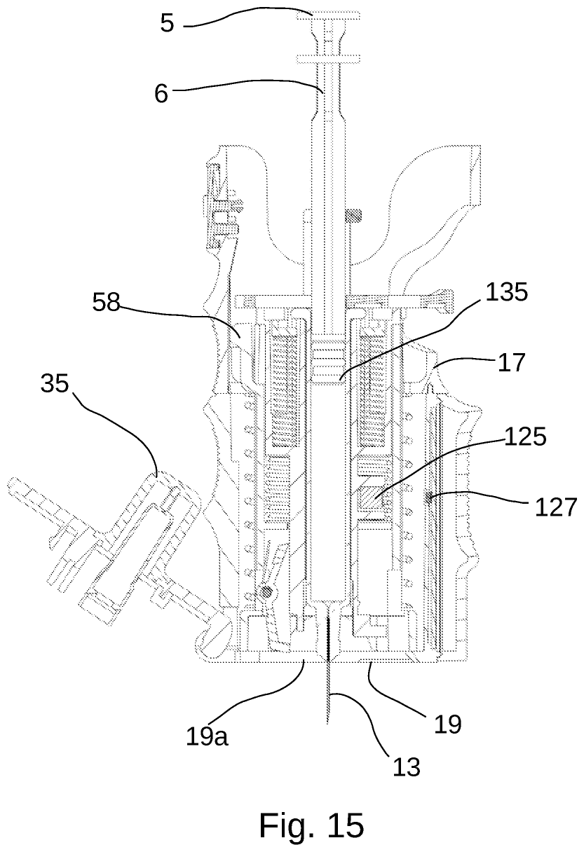

As has been described above, once the needle cap holder (35) has temporarily removed the needle cap (16) from needle (13), the inserter device (1) can be moved into the injection ready position. The manner by which to achieve this has been described in detail above. It should be noted that the biasing force released from the pent up energy store embodied by the outer biasing spring (60) when the release button (133) is pressed can be sufficient to cause the distal ends of the inner slider body to contact the distal end of the inserter body and produce an audible sound. Such a sound can therefore indicate to the user that the injection ready position has been attained. Alternatively, the injection ready position can be detected by the position sensor (137) in relation to the signals, e.g, magnetic vector, or magnetic field strength, produced by the position signaling element (135) and detected by the position sensor when the automatic inserter device (1) is in the injection ready position. The relative positions of the components of the inserter device in the injection ready position is illustrated in FIG. 15. In the injection ready position, at least a distal part of the pre-filled syringe (2) extends beyond a distal end (19) of the inserter body (17), and more specifically, the needle (13) extends beyond the distal end (19) of the inserter body (17). The degree or length to which the needle (13) extends beyond the end of the inserter body (17) is designed in such a way as to avoid excessive trauma to the user, and to minimize any associated pain due to the depth of penetration of the needle at the administration site. It should be noted here that the term “injection-ready” only signifies that the pre-filled syringe is in a suitable position in which to effect the injection of product contained within the syringe chamber (4), not that an injection has actually been carried out, or activated. The syringe plunger and shaft (6) must still be depressed by the user to expel the product from the chamber into the site of administration. This is carried out as a subsequent operation to moving the slidable carriage assembly (50) and pre-filled syringe (2) inserted therein into the injection-ready position.

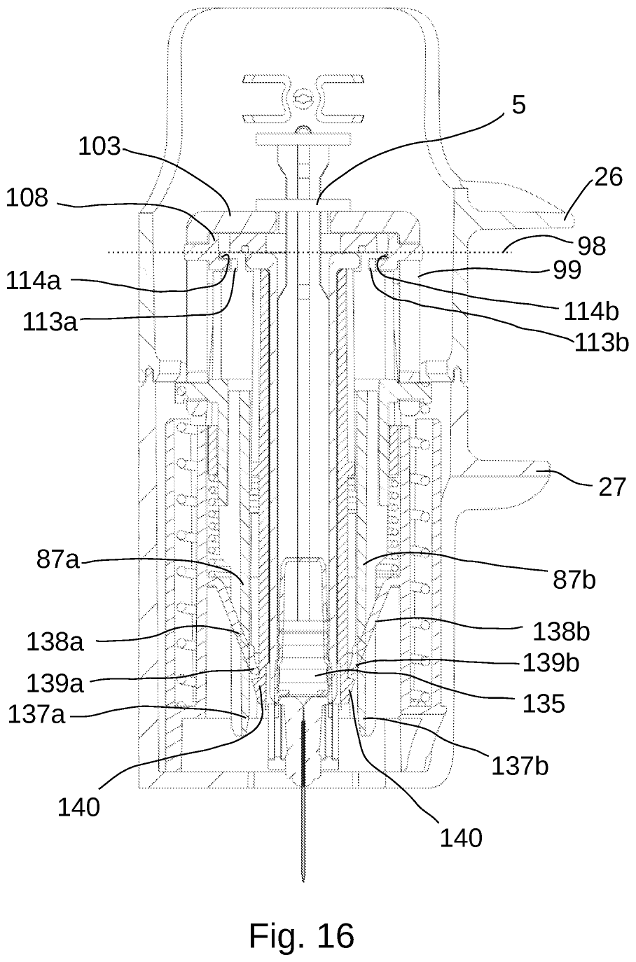

Injection of the substance contained with the chamber (4) is effected by pressing on the plunger head (5) of the plunger shaft (6). As the plunger head (5) moves in a distal direction, and ejects the substance contained within the chamber through the needle (13), the plunger head (5) comes into contact with the cradle (105) of the support collar (99), and the support collar translates in a proximal direction as well, due to the interaction of the slots (Ila, 111b) of the support collar legs (100a, 100b) and the locking plate spigots (109a, 109b). When the plunger has completed its maximum allowed distance of travel, i.e. when a distal plunger plug (135) attached to the distal end of the plunger shaft (6) has reached the distal bottom of the chamber (4) of the syringe body (3), the plunger head comes into contact with a proximal-facing surface (136) of the support cradle (105) to prevent any further distal movement of the plunger shaft (6) and plunger head (5), which might otherwise cause damage to the inner and outer slider bodies (46, 51). FIG. 16 shows the relative positions of the components of the automatic inserter device (1) once injection has been completed.

After injection has been completed, the inner slider body (46) is moved together with the locking plate (108) in a proximal direction to a third, syringe-retracted position. The syringe retracted position corresponds to an axial position along the longitudinal axis of the inserter bore (20) in which the needle no longer extends beyond the distal open end (19) of the inserter bore (20). In this configuration, were the syringe to be thrown away with the inserter body, then the third position could be a final position into which the syringe would be moved, but advantageously, the automatic inserter is also configured to be re-used, and therefore, the third position is only an intermediate position, the aim of which is to safeguard the inserter device (1) and the user against accidents from a potentially exposed needle after injection has been completed. In this retracted position, the needle (13) of the PFS (2) is entirely contained with the bore (20) of the inserter body (17).

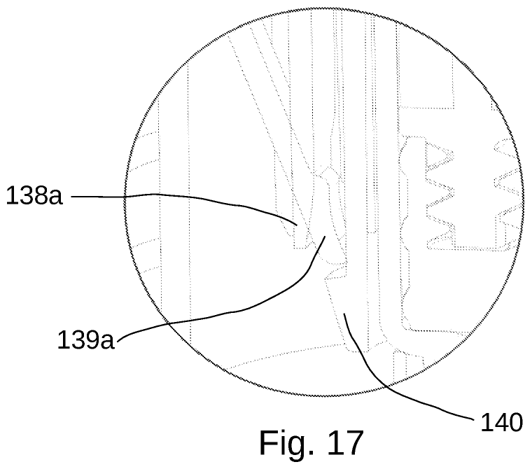

Movement of the syringe and needle into the needle-retracted position is achieved as follows and is illustrated by FIGS. 16 and 17. The user presses down on the transverse bar (103) of the support collar (99). The support collar legs (100a, 100b) are connected to the caliper (92) via the diametrically opposed spars (93a, 93b) and the distal movement applied to the caliper body (92) through the support collar legs (100a, 100b) causes in turn the caliper legs (87a, 87b) to move in a distal direction, whereupon the distal ends (137a, 137b) of the caliper legs (87a, 87b) come to bear on, and deform, corresponding elastically deformable spars (138a, 138b) which extend obliquely in a distal direction from the outer slider body (51) into the bore (56). The elastically deformable spars (138a, 138b) have distal feet (139a, 139b) which, before any elastic deformation, abuttingly engage with an annular seating rim (140) extending in a proximal direction from the distal end of the inner slider body (46). The interaction of the distal feet (139a, 139b) and annular seating rim (140) prevent the inner slider body (46) and outer slider body from moving independently, one from the other, in the proximal direction. As the distal ends (137a, 137b) of the caliper legs (87a, 87b) are moved in a distal direction, they engage slidingly with the elastically deformable spars (138a, 138b), causing the spars to be moved away elastically from their abutting engagement with the seating rim (140), thereby releasing the inner slider body (46) from being retained by the outer slider body (51). The inner biasing spring (72) of the outer slider body (51) now urges the inner body (46) in a proximal direction, until the seating rim (140) comes into abutting contact with a distal-facing surface (141) of the radially inwardly radially projecting shoulder (71) of the outer slider body (51). The total distance moved by the inner slider body in the proximal direction at this step is approximately 12 mm. The urging force of the inner biasing spring (72) can be configured to produce an audible sound as the seating rim abuts against the distal-facing surface of the radially inwardly extending shoulder (71), alerting the user to the fact that the needle and syringe body have been retracted. Alternatively, the movement of the inner body (46), which houses the position signaling element (135), will be detected by the position sensor (137) as an intermediate position which is proximal compared to the initial rest position, and distal of the armed position, and correspondingly proximal of the injection ready position, and can thus be classified accordingly by the micro-controller and or processing unit. It should be noted that the distal end (14) of the needle (13) of the PFS (2) is positioned at the correct distance from the distal end (19) of the inserter body to allow it to be coiffed once more by the needle cap, as explained hereafter.

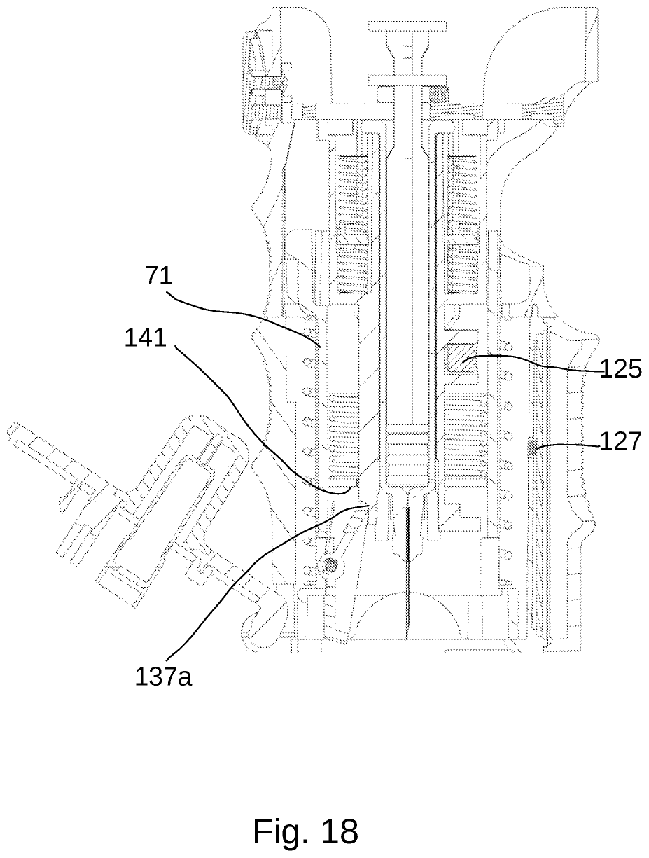

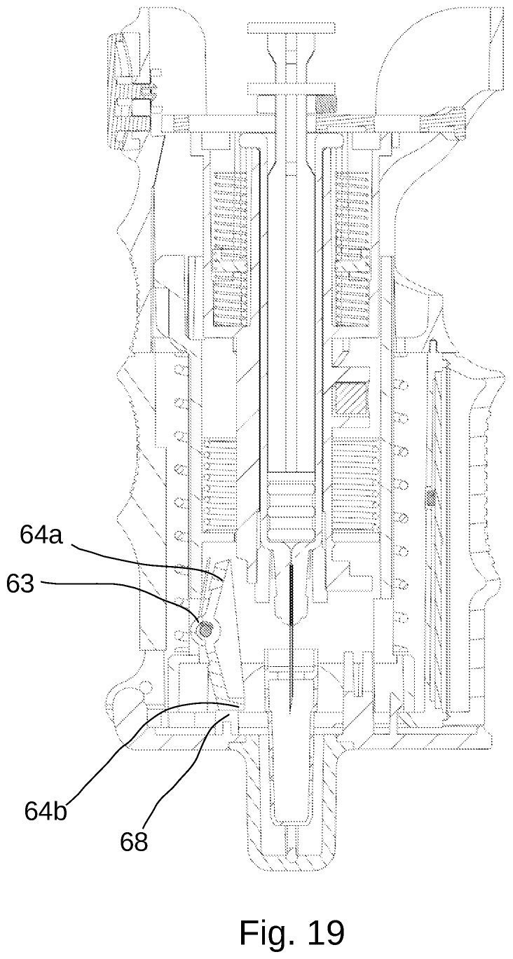

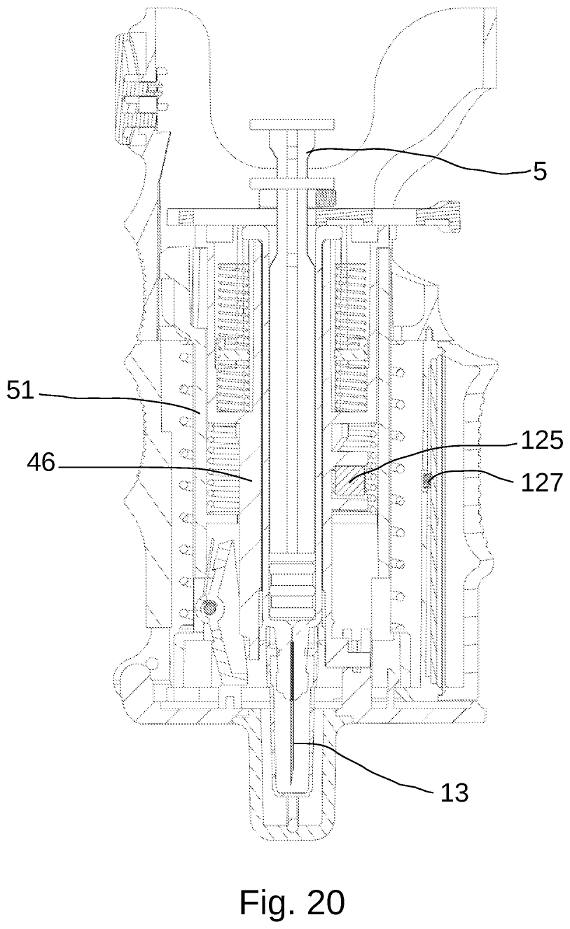

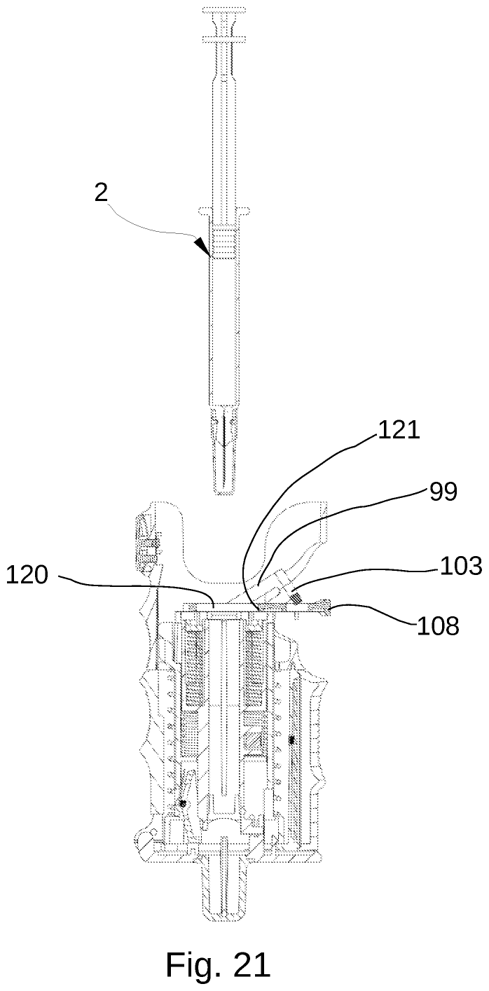

An additional safety measure, as illustrated by FIGS. 19 and 20, is provided in form of the lever arm safety lock (63). As has been explained elsewhere in the present specification, the lever arm lock (63) has a biased proximal lever arm locking member (64a) which is normally counter-biased by the presence of the needle cap holder (35), when the latter is in the closed position and bears against the distal locking arm member (64b). However, when the needle cap holder (35) is in the open position, for example as shown in FIG. 18, the proximal arm locking member (64a) is biased by the lever arm biasing spring (67) to project into the bore (56) of the outer slider body (51) and engage with a distal end (137a) of one of the caliper legs (87a). This prevents accidental distal movement of the legs (87a, 87b) and the corresponding risk of pushing the needle out of the retracted position into a potentially stick injury inducing position before the needle cap holder (35) and corresponding needle cap (16) temporarily held therein has been moved back into the closed position.

As illustrated in FIG. 19, once the needle and syringe have reached the syringe-retracted position, the needle cap holder (35) can be moved via rotation around the articulation (37) from the open position to the closed position, in which the needle cap holder closes the previously open distal end of the inserter device body (17). Doing so will correspondingly move the needle cap (16) back into position for it to remount onto the distal end (14) of the needle (13). Once back in the closed position, the needle cap holder (35) engages once again with the distal locking arm member (64b) via the contact spigot (68), biasing said arm member to rotate around the fulcrum pivot point (65), thereby moving the proximal locking arm member (64a) back towards the outer slider body (51) and away from blocking any distal movement of the caliper legs (87a, 87b).

As illustrated in FIG. 20, it is now once again possible to move the caliper legs (87a, 87b) in a distal direction, pressing down on the transverse bar (103) of the support collar (99) will move the inner slider body (46), and caliper (92) in a distal direction, along with the locking plate (108) into a syringe-release position. Engaging the syringe release position moves the inner (46) and outer (51) slidable bodies back into a position allowing the now empty or expended syringe to be removed safely from the inserter bore (20). In particular, the inner projecting member (43) is moved distally, when it forms part of the inner slider member (46) into frictional engagement contact with the elastically deformable friction grip member (45) of the needle cap holder (35). This position is illustrated in FIGS. 19 and 20, in which it can be seen that inner (46) and outer (51) slider bodies have returned to the initial rest position. Such a rest position can be accordingly detected and registered by the microelectronic circuitry using the position signaling element (125) and the position sensor (127). Additionally, in order to release the PFS (2) from the automatic inserter device (1), the locking plate (108) is moved laterally once again in a reverse direction to that used to move the locking plate into the locking position, thereby removing any engagement between the locking plate grooves (114a, 114b) and the proximal lip (115) of the inner slider body (46), but equally also thereby moving the support collar (99) and cradle (105) from the deployed, or vertical position, back to the collapsed, or horizontal position. At the same time, the subsidiary opening (121) of the locking plate is moved away from the plunger shaft (6), to the larger dimensioned main locking plate opening (120), thereby allowing safe withdrawal of the now empty syringe and capped needle from the automatic inserter device (1) via the proximal end (18) of the inserter body (17).

1) An automatic inserter for a pre-filled injection syringe system comprising:
an elongated inserter body having a proximal extremity and a distal extremity, and a longitudinal bore extending through the elongate inserter body from the proximal extremity to the distal extremity, the longitudinal bore having a central longitudinal axis, wherein the elongated inserter body is dimensioned and configured to receive a pre-filled injection syringe introduced into said bore via the proximal extremity of the inserter body, and is further configured and dimensioned to prevent the pre-filled injection syringe from exiting the longitudinal bore via the distal extremity; and
wherein the automatic inserter further comprises an articulated cap holder connected to the distal extremity of the inserter body via a rotatable articulation, and the needle cap holder is configured to be moved via rotation of the articulation about an axis of rotation of the articulation, from a first, closed position, in which the needle cap holder receives a needle cap removably mounted onto an injection needle of the pre-filled injection syringe when the pre-filled injection syringe is received in the inserter body prior to injection, to a second, open position in preparation for an injection step, in which the needle cap holder holds the needle cap at a location removed from the pre-filled injection syringe, whilst still being connected to the inserter holder body through the rotatable articulation.

2) The automatic inserter according to claim 1, wherein the rotatable articulation has an axis of rotation which lies orthogonal to the central longitudinal axis of the inserter body.

3) The automatic inserter according to claim 1, wherein the articulated needle cap holder is configured to be moved, after an injection has completed, from the second position back to the first position, via rotation of the needle cap holder about the axis of rotation of the articulation, thereby repositioning the needle cap back onto the injection needle of the pre-filled injection syringe.

4) The automatic inserter according to claim 1, wherein the articulated needle cap holder comprises an elastically deformable friction grip surface configured to engage with, and grip, an outer surface of the needle cap.

5) The automatic inserter according to claim 4, wherein the friction grip surface comprises an elastomer.

6) (canceled)

7) The automatic inserter according to claim 1 wherein the articulated needle cap holder comprises, in the first, closed position, an elastically deformable friction grip member configured to elastically deform around, and releasably engage with, an inner projecting member located within the bore of the inserter body, and wherein, in the second position, the friction grip member of the needle cap holder is free from engaging contact with the inner projecting member.

8) The automatic inserter according to claim 7, wherein the elastically deformable friction grip member is formed as an extension of a body of the articulated needle cap holder, and the extension projects into the bore of the inserter body.

9) The automatic inserter according to claim 1, wherein the articulated needle cap holder, in the first position, covers a distal opening of the longitudinal bore of the inserter body and in the second position, leaves the distal opening of the inserter body bore open.

10) (canceled)

11) An automatic inserter for a pre-filled injection syringe system comprising:
an elongated inserter body having a proximal extremity and a distal extremity, and a longitudinal bore extending through the elongate body from the proximal extremity to the distal extremity, the longitudinal bore having a central longitudinal axis, wherein the elongated inserter body is dimensioned and configured to receive a pre-filled injection syringe introduced into said bore via the proximal extremity of the inserter body, and is further configured and dimensioned to prevent the pre-filled injection syringe from exiting the longitudinal bore via the distal extremity;
wherein the inserter body comprises a slidable syringe body carriage assembly located within the bore which is configured to receive and at least partially surround a syringe body of the pre-filled injection syringe, wherein the slidable carriage assembly comprises a translation system configured to translate the slidable carriage assembly and the syringe body received therein, along the central longitudinal axis of the inserter body bore, from one axial position to at least one other axial position which is axially distant from said one axial position.

12) (canceled)

13) The automatic inserter according to claim 11, wherein the inserter includes a syringe plunger locking plate configured and adapted to be moved from an unlocked position to a locked position, wherein in the unlocked position, the locking plate is disengaged from a syringe plunger of the pre-filled injection syringe, and in the locked position, the locking plate engages the syringe plunger and prevents axial movement of the syringe plunger relative to the locking plate.

14) The automatic inserter according to claim 13, wherein the locking plate is configured to be moved orthogonally to the syringe plunger and a proximal end of the syringe body from the unlocked position, to the locked position.

15) The automatic inserter according to claim 13, wherein in the unlocked position an end of the locking plate extends outside the inserter body, and in the locked position, said end is contained completely within the longitudinal bore of the inserter body.

16) The automatic inserter according to claim 11, wherein the slidable syringe body carriage assembly comprises a first axially movable cylinder located co-axially within the longitudinal bore, and a second axially movable cylinder located co-axially within the longitudinal bore of the inserter body, and generally co-axially within a bore of the first movable cylinder.

17) The automatic inserter according to claim 16, wherein, the first axially movable cylinder and the second axially movable cylinder are configured to be moved axially within the longitudinal bore independently one from the other.

18) The automatic inserter according to claim 16, wherein, the first axially movable cylinder and the second axially movable cylinder are configured to be moved axially within the longitudinal bore together.

19) (canceled)

20) The automatic inserter according to claim 11, wherein the one axial position is a resting position and the slidable carriage assembly is movable from the one axial position to the at least one other axial position to move the pre-filled injection syringe in a proximal direction to an armed position to arm the pre-filled syringe for a subsequent axial acceleration prior to an injection step.

21) The automatic inserter according to claim 11, wherein the at least one other axial position is an injection-ready position in which at least a distal part of the pre-filled syringe extends beyond a distal end of the inserter body.

22) The automatic inserter according to claim 20, wherein the slidable syringe body carriage assembly comprising a first axially movable cylinder and a second axially movable cylinder located coaxially within the longitudinal bore and the second axially movable cylinder located within the first movable cylinder and the first and second axially movable cylinders are moved together in the proximal direction into the armed position simultaneously with a syringe plunger locking plate.

23) (canceled)

24) The automatic inserter according to claim 22, wherein the inserter includes a needle cap holder movably coupled to the inserter body and movable between a first, closed position, to a second, open position and wherein the needle cap holder is movable between the closed position to the open position when the first axially movable cylinder, second axially movable cylinder, and the locking plate are in the armed position.

25) The automatic inserter according to claim 21 wherein the slidable syringe body carriage assembly comprises a first axially movable cylinder and a second axially movable cylinder located coaxially within the first axially movable cylinder and the first and second axially movable cylinders are configured to be moved together in a distal direction into the injection-ready position simultaneously with a locking plate

26) The automatic inserter according to claim 25, wherein the second axially movable cylinder of the slidable syringe body carriage assembly is configured to be moved together with the locking plate in a proximal direction to a syringe-retracted position and in a distal direction to the syringe-release position.

27) (canceled)

28) (canceled)

29) The automatic inserter according to claim 11 wherein the slidably carriage assembly includes first and second axially movable cylinders where the second axially movable cylinder is coaxially located within the first axially movable cylinder and the inserter body includes a syringe plunger locking plate and the locking plate comprises a locking means configured to releasably engage with a proximal end of the second axially movable cylinder of the slidably carriage assembly.

30) The automatic inserter according to claim 13, wherein the locking plate comprises a plunger shaft support collar extending in a proximal direction from the locking plate to engage in sliding contact with at least a portion of the plunger shaft, the support collar being configured to move from a collapsed state to a deployed state or from a deployed state to a collapsed state.
